(19) United States
(12) Patent Application Publication
(10) Pub. No.: US 2026/0265807 A1
(43) Pub. Date: Sep. 10, 2026

(54) METHODS AND COMPOSITIONS FOR NUCLEIC ACID SEQUENCING

(71) Applicant: Broken String Biosciences Limited, Little Chesterford Saffron Walden (GB)

(72) Inventors: Felix DOBBS, Cambridge (GB); Simon REED, Cambridge (GB); Patrick VAN EIJK, Cardiff (GB)

(21) Appl. No.: 18/876,531

(22) PCT Filed: Jun. 21, 2023
(86) PCT No.: PCT/EP2023/066881

§ 371 Date: Dec. 18, 2024

(30) Foreign Application Priority Data
Jun. 22, 2022 (GB) 2209189.6

(51) Int. Cl. C12Q 1/6806; C12Q 1/6855

(52) U.S. Cl. CPC C12Q 1/6806; C12Q 1/6855

(57) ABSTRACT

In an aspect, the invention relates to methods of library preparation and compositions suitable for use in methods of library preparation. The invention also relates to methods of sequencing and to uses of libraries in sequencing.

### FIELD OF THE INVENTION

In an aspect, the invention relates to methods of library preparation and compositions suitable for use in methods of library preparation. The invention also relates to methods of sequencing and to uses of libraries in sequencing.

### BACKGROUND

Current nucleic acid sequencing methods, such as next generation sequencing (NGS), enable sequence information to be derived from extremely large samples. For instance, it is possible to gather genome-wide sequence information. However, both sample preparation methods and sequencing methods are error prone. This is particularly problematic for applications that require small changes to be detected in a large sample, for example the detection of single base pair changes in a genome, because even a very low error rate can affect the outcome.

The need for the detection of rare mutations is growing in light of the increasing use of gene editing technologies, such as CRIPSR-Cas9 and the like. CRISPR genome editing uses a synthetic guide RNA to target Cas9 enzyme—the nuclease that acts as the genetic scissors—to a specific site in the genome where a genetic change is required. Genome editing relies on the accurate targeting of these sites to generate small insertions or deletions to manifest genetic change. However, this involves the introduction of a DNA Double Strand Break (DSB) in the DNA molecule. Such breaks can have detrimental effects on human health and can cause cancer depending on their location and the cell's ability to repair them. The system is highly accurate in its targeting, however, secondary, so-called off-target sites in the genome can also be targeted unintentionally during the editing process. These positions often resemble the target sequence but in ways that are currently not fully understood. Indeed, in silico off-target prediction—based solely on the guide RNA sequence—is often not sufficiently accurate to reveal all experimentally detected off-target sites. This is required to improve guide design and prevent off-target editing. It is important to note that the specificity of guide RNAs is highly variable, which has important implication for their safe use in gene therapies. Indeed, off-target sites can receive breaks and/or mutations throughout the genome, posing an important and inherent risk of genome editing in general. This in turn, presents a serious challenge to current legacy cell-based methods for genotoxic risk assessment that assess the effects of chemicals on the stability of the genome. However, genome editing uses a novel class of targeted biologicals that present a needle-in-the-haystack type of problem: how to recognise rare off-target editing events in a complex genome when they are not predictable by sequence alone. The off-target problem has been exacerbated by CRISPR-Cas9 genome editing because the off-targets introduced are now so rare that they cannot be detected by the current cell-based methods.

To assess the long-term impact of these off-target breaks it is important to measure their mutational outcomes determined by their accurate repair. Existing methods, such as amplicon-seq, have a reported mutation detection limit of ~1 in 10³. The sensitivity of amplicon-seq is therefore insufficient for the detection of rare mutations at novel, low-frequency off-target sites.

Schmitt et al. discloses a method that aims to detect ultra-rare mutations by next-generation sequencing (PNAS, Sep. 4, 2012, vol. 109, no. 36, pages 14508-14513). Further described in detail by Kennedy, S. R., et al. (Detecting ultralow-frequency mutations by Duplex Sequencing. Nat Protoc, 2014. 9(11): p. 2586-606). Schmitt et al. discloses independently tagging and sequencing each of the two strands of a DNA duplex. The disclosed method requires appending a double-stranded, randomized Duplex Tag sequence to a sequencing adapter by copying a degenerate sequence in one strand of the adapter with DNA polymerase. A similar method includes NanoSeq disclosed in Abascal et al. (Somatic mutation landscapes at single-molecule resolution. Nature, 593, 405-410 2021) describing an optimised version of the BotSeqS method that applies enzymatic fragmentation and a modified end-repair procedure to improve error-corrected sequencing using UMI tags as described above. However, detecting mutations genome-wide with these methods is currently prohibitively expensive for larger genomes because this method requires a high coverage (>10⁴ fold) (Kennedy, S. R., et al. 2014), restricting its use to targeted sequencing of off-targets that have already been identified, such as by a DSB-detection method. Moreover, the selection of restriction enzyme for DNA fragmentation used in NanoSeq only provides partial coverage of the genome. Sonication and exonuclease blunting have been proposed as an alternative, but this approach would suffer from the same excessive coverage requirements described previously.

WO 2013/142389 A1 discloses methods that aim to lower the error rate of massively parallel DNA sequencing using duplex consensus sequencing. WO 2013/142389 A1 discloses the formation of a library by the ligation of adapters to DNA to result in three products (referred to as “Product I”, “Product II”, and “Product III”).

There is a need for further methods capable of producing error-corrected sequence information. In particular, there is a need for methods capable of providing unbiased and independent determination of gene editing-induced mutations close to background level at low-frequency off-target sites and throughout the genome.

### SUMMARY OF THE INVENTION

In an aspect, there is provided a method of library preparation for nucleic acid sequencing, the method comprising:
a) providing a plurality of nucleic acids;
b) exposing the plurality of nucleic acids to a non-hairpin adapter under conditions conducive to ligation;
c) fragmenting the plurality of nucleic acids; and
d) exposing the plurality of nucleic acids to a hairpin adapter under conditions conducive to ligation, or exposing the plurality of nucleic acids to conditions capable of forming a hairpin at an end of a nucleic acid molecule;
wherein steps b) and d) are performed separately.

The plurality of nucleic acids is fragmented after the first adapter ligation step and before, or as a part of, the second adapter ligation step. The first ligation step is the first of step b) or step d) to be performed. The second ligation step is the second of step d) or step b) to be performed. Thus, the first ligation step is either: i) step b) where step d) is the second ligation step or ii) step d) where step b) is the second ligation step.

The steps may be performed sequentially and in the order a), b), c), d). The steps may be performed sequentially and in the order a), d), c), b). The steps may be performed in the order step a), step b), and combined steps c) and d). The steps may be performed in the order step a), step d), and combined steps c) and b).

The non-hairpin adapter may comprise a sequence that is at least partially complementary to a first primer that is immobilised to a substrate. The sequence that is at least partially complementary to a first primer that is immobilised to a substrate may comprise at least 5, 10, 15, 16, 17 18, 19, 20, or all 21 bases of SEQ ID NO: 1 or at least 5, 10, 15, 16, 17 18, 19, 20, 21, 22, 23, or all 24 bases of SEQ ID NO: 3.

The non-hairpin adapter may be a Y-adapter. The Y-adapter may comprise a first strand comprising a sequence that is at least partially complementary to a first primer immobilised to a substrate; and a second strand comprising a sequence that is identical to at least a region of a second primer. The sequence that is identical to at least a region of a second primer may comprise at least 5, 10, 15, 16, 17 18, 19, 20, 21, 22, 23, or all 24 bases of SEQ ID NO: 2 or at least 5, 10, 15, 16, 17 18, 19, or all 20 bases of SEQ ID NO: 4. The non-hairpin adapter is a Y-adapter that comprises a first strand comprising, in the 5′ to 3′ direction, a first hybridisation site to which a first sequencing primer can bind, and a sequence that is at least partially complementary to a first immobilised primer; and a second strand comprising, in the 5′ to 3′ direction, a sequence that is identical to a region of a second immobilised primer and a second hybridisation site to which a second sequencing primer can bind.

The non-hairpin adapter may comprise a 5′ and/or a 3′ protective feature. The non-hairpin adapter may comprise a first strand comprising a 3′ protective feature and a second strand comprising a 5′ protective feature.

The non-hairpin adapter may be a Y-adapter that comprises: a first strand comprising, in the 5′ to 3′ direction, a first hybridisation site to which a first sequencing primer can bind, a sequence that is at least partially complementary to a first immobilised primer, and a 3′ protective feature; and a second strand comprising, in the 5′ to 3′ direction, a 5′ protective feature, a sequence that is identical to at least a region of a second primer, and a second hybridisation site to which a second sequencing primer can bind.

The plurality of nucleic acids may be DNA or genomic DNA (gDNA).

The method may further comprise: e) contacting the plurality of nucleic acids to a substrate comprising a first immobilised primer under conditions suitable for hybridisation of the first immobilised primer to complementary nucleic acids; wherein the non-hairpin adapter comprises a sequence that is at least partially complementary to the first immobilised primer. Optionally no nucleic acid amplification step is performed prior to step e). The substrate may be a flow cell or a bead.

The non-hairpin adapter may comprise a sequence that is identical to at least a region of a second primer and the second primer is immobilised to the substrate. The first and second immobilised primers may be capable of acting as forward and reverse primers for bridge amplification, and wherein the method may comprise bridge amplification.

The method may further comprise: f) obtaining sequence information for any nucleic acids that hybridised to the substrate in step e).

After steps a), b), c), and d) have been performed, the method may further comprise obtaining sequence information from the prepared library.

In another aspect, there is provided a nucleic acid library obtained or obtainable by a method of the present disclosure.

In another aspect, there is provided a nucleic acid library comprising a target nucleic acid with a non-hairpin adapter ligated to one end and a hairpin at the other end, wherein the nucleic acid library comprises less than 99.9%, 99%, 95%, 90%, 80%, 70%, 60%, 50%, 40%, 30%, 20%, 10%, 5%, 3%, 1%, 0.1%, or 0.01% by mass, or none, of target nucleic acid with a non-hairpin adapter ligated to both ends.

In another aspect, there is provided a method of sequencing, wherein the method comprises obtaining sequence information for nucleic acids within a library of the present disclosure.

In another aspect, there is provided a method of obtaining sequencing information, wherein the method comprises:
1) contacting a library of the present disclosure to a substrate comprising a first immobilised primer under conditions suitable for hybridisation of the first immobilised primer to complementary nucleic acids; and
2) obtaining sequence information for any nucleic acids that hybridised to the substrate in step 1).

In another aspect, there is provided the use of a nucleic acid library of the present disclosure, or a nucleic acid library obtained or obtainable by a method of the present disclosure, in a nucleic acid sequencing method.

In another aspect, there is provided a method of library preparation for nucleic acid sequencing, the method comprising:
i) providing a plurality of nucleic acids;
ii) exposing the plurality of nucleic acids to a non-hairpin adapter under conditions conducive to ligation; and
iii) exposing the plurality of nucleic acids to a hairpin adapter under conditions conducive to ligation, or exposing the plurality of nucleic acids to conditions capable of forming a hairpin at an end of a nucleic acid molecule;
   wherein the nucleic acids are not amplified during preparation of the library. Optionally, wherein the method further comprises:
iv) contacting the plurality of nucleic acids to a substrate comprising a first immobilised primer under conditions suitable for hybridisation of the first immobilised primer to complementary nucleic acids;
   wherein the non-hairpin adapter comprises a sequence that is at least partially complementary to the first primer that is immobilised to the substrate; and
   wherein the nucleic acids are not amplified prior to step iv).

The non-hairpin adapter may comprise a sequence that is identical to at least a region of a second primer and the second primer is immobilised to the substrate. The first and second immobilised primers may be capable of acting as forward and reverse primers for bridge amplification, and wherein the method may comprise bridge amplification.

The method may comprise obtaining sequence information for any nucleic acids that hybridised to the substrate in step iv). Steps ii) and iii) may be performed separately, and wherein a fragmentation step may be performed after step ii) and before step iii) or after step iii) and before step ii).

In another aspect, there is provided a nucleic acid library obtained or obtainable by the above methods. In another aspect, there is provided a method of sequencing, wherein the method comprises obtaining sequence information for nucleic acids within said library. In another aspect, there is provided a method of obtaining sequencing information, wherein the method comprises: 1) contacting said library to a substrate comprising a first immobilised primer under conditions suitable for hybridisation of the first immobilised primer to complementary nucleic acids; and 2) obtaining sequence information for any nucleic acids that hybridised to the substrate in step 1). Also provided is the use of said nucleic acid library, or a nucleic acid library obtained or obtainable by said method, in a nucleic acid sequencing method.

### BRIEF DESCRIPTION OF THE DRAWINGS

FIG. 1. Exemplary illustration of an embodiment of “DEDUCE-seq” (DuplEx Determination by Unbiased flow Cell Enrichment and sequencing).

FIG. 2. Exemplary illustration of sequencing. Mutations are read twice due to the linking of the DNA duplex.

FIG. 3. Exemplary illustration of binding to the substrate (in this case, a flow cell). DNA from Y-adaptor ligated fragments bind to the flow cell and hairpin-only ligated fragments are washed away.

FIG. 4. Exemplary overview of an embodiment of the methods of the present disclosure.

FIG. 5. DEDUCE-seq library preparation for Pilot-1; DNA size distribution and quantification. 1) Left Panel—Genomic DNA was size selected to ~100-500 bp (black trace) by removing DNA>300 bp (gray trace). 2) Middle panel—Ligated (black trace) and resonicated DNA (grey trace) were tested by gel electrophoresis. 3) Right-panel—Post-sonication (black trace) and final library DNA of a DEDUCE-seq library is shown here.

FIG. 6. Distribution of HP sequences throughout the length of 9.5M Forward Reads (Left Panel) and Reverse Reads (Right Panel) from Pilot-1.

FIG. 7. DEDUCE-seq library discordant read pairs. Shown here are 3 examples of discordant, parallel reads pairs derived from a DEDUCE-seq library. The leftmost read pair (F1F2, reads are stacked) consists of left alignment (read F1, SAM flag 67) and right alignment (read F2, SAM flag 131) both mapped to the forward stand. The right read pair (R2R1) consists of left alignment (read R2, SAM flag 115) and right 2 (read R1, SAM flag 179). Read details for the second reverse alignment on the right are not shown.

FIG. 8. DEDUCE-seq library preparation for Pilot-2; DNA size distribution and quantification. 1) Left Panel—Genomic DNA was size selected to ~100-500 bp (black trace) by removing DNA>300 bp (gray trace). 2) Middle panel—Y-adapter ligated (black trace) and resonicated DNA (grey trace) were tested by gel electrophoresis. 3) Right-panel—Final library DNA of a DEDUCE-seq sample is shown here.

FIG. 9. Distribution of hairpin sequence in reads R1 and R2 of DEDUCE-seq library molecules sequenced in Pilot-2.

### DETAILED DESCRIPTION

The inventors provide herein techniques for preparing nucleic acid libraries that are suitable for generating error-corrected sequencing data. When the nucleic acid libraries of the present disclosure are sequenced, sequencing information is provided for both strands of a nucleic acid duplex. This allows the correction of errors, including those that have been introduced via library preparation or that arise as a result of the sequencing.

In a first aspect, there is provided a method of library preparation for nucleic acid sequencing, the method comprising:
a) providing a plurality of nucleic acids;
b) exposing the plurality of nucleic acids to a non-hairpin adapter under conditions conducive to ligation;
c) fragmenting the plurality of nucleic acids; and
d) exposing the plurality of nucleic acids to a hairpin adapter under conditions conducive to ligation or exposing the plurality of nucleic acids to conditions capable of forming a hairpin at an end of a nucleic acid molecule;
wherein steps b) and d) are performed separately.

Steps b), and d) of the method of the first aspect are performed separately, and so the non-hairpin adapter and the hairpin adapter are not ligated to the nucleic acids as a part of the same reaction. In other words, steps b) and d) are not performed simultaneously.

However, as discussed further herein, adapter ligation and fragmentation steps may be performed simultaneously, in combination, or concurrently. For instance a tagmentation step may be used to both ligate an adapter and to fragment the plurality of nucleic acids. As such, steps b) and c), or steps d) and c), may be performed simultaneously, in combination, or concurrently.

For all embodiments, the plurality of nucleic acids is fragmented after the first adapter ligation step and before, or as a part of, the second adapter ligation step.

Steps b), c), and d) of the method of the first aspect may be performed sequentially. The steps of the method may be, but need not be, performed in the order a), b), c), and then d). In particular, steps b) and d) may be swapped such that the order may be a), d), c), and then b). The fragmenting step (step c)) may be performed in-between the ligation of one type of adapter and the ligation of the other type of adapter. The fragmenting step is performed at the time of, or before, the second ligation of an adapter.

As used herein, the term “sequentially” means that the steps are not simultaneous. However, sequential steps need not be consecutive and additional steps may be performed in-between explicitly recited steps.

In other embodiments, the steps may be in the order: step b) and then steps c) and d) concurrently, in combination, or simultaneously. The steps may be in the order: step d) and then steps c) and b) concurrently, in combination, or simultaneously.

Due to the fact that steps b) and d) are performed separately, the non-hairpin adapter and the hairpin adapter are not ligated to the nucleic acids at the same time or as part of the same step. For embodiments where a hairpin is formed, this process does not take place at the same time or as part of the same step as the ligation of the non-hairpin adapter.

Thus, in an embodiment, there is provided a method of library preparation for nucleic acid sequencing, the method comprising:
a) providing a plurality of nucleic acids;
b) exposing the plurality of nucleic acids to a non-hairpin adapter under conditions conducive to ligation to generate a first library;
c) fragmenting the first library; and
d) exposing the fragmented first library to a hairpin adapter under conditions conducive to ligation to generate a second library, or exposing the plurality of nucleic acids to conditions capable of forming a hairpin at an end of a nucleic acid molecule to generate a second library.

The fragmentation of the first library and the generation of the second library may be sequential or simultaneous steps.

In an alternative embodiment, there is provided a method of library preparation for nucleic acid sequencing, the method comprising:
a) providing a plurality of nucleic acids;
b) exposing the plurality of nucleic acids to a hairpin adapter under conditions conducive to ligation to generate a first library, or exposing the plurality of nucleic acids to conditions capable of forming a hairpin at an end of a nucleic acid molecule to generate a first library;
c) fragmenting the first library; and
d) exposing the fragmented first library to a non-hairpin adapter under conditions conducive to ligation to generate a second library. The fragmentation of the first library and the generation of the second library may be sequential or simultaneous steps.

A nucleic acid library is a collection or plurality of nucleic acids to which at least one type of adapter has been ligated.

The libraries provided by the methods of the first aspect have a reduced amount of sequencable nucleic acids that would generate un-error-correctable sequence information associated only with one strand of a duplex. Such undesired nucleic acids include those comprising, for instance, a non-hairpin adapter ligated to both ends of the nucleic acid. This is advantageous because it eliminates the need for enrichment and/or amplification prior to sequencing or prior to substrate-based steps. In addition, the quality of the library is improved. The ability to derive sequence information from a library without the need for an amplification step prior to sequencing, for instance prior to contacting the library with a substrate such as a flow cell, is advantageous because such steps can, themselves, introduce mutations and bias. In some embodiments, the libraries provided by the methods of the first aspect have a significantly reduced presence of sequencable nucleic acids that would generate un-error-correctable sequence information associated only with one strand of a duplex, for instance a reduction that is sufficient for the library to be sequenced on a substrate without the need for enrichment or amplification prior to application to the substrate. In some embodiments, it is desirable to generate libraries of the first aspect that do not comprise sequencable nucleic acids that would generate un-error-correctable sequence information associated only with one strand of a duplex. However, any reduction in their presence, for instance such that amplification is no longer required, is advantageous. Prior art methods leading to libraries containing undesired products are disclosed in, for instance, WO 2013/142389 A1.

The provision of a plurality of nucleic acids may be performed as the first step of the method. This step may comprise the purification of nucleic acids, such as DNA, from a sample. The nucleic acids purified or isolated from the sample may be genomic DNA (gDNA). Thus, the provision of a plurality of nucleic acids may be the provision of DNA or gDNA molecules to be sequenced, which may be referred to as target nucleic acids. The sample may be a biological sample, such as a sample obtained from a patient or a sample obtained from biological cells. The sample may be a tissue sample, a sample of a biological fluid, a cell line, or any other suitable sample. The sample may comprise normal, neoplastic, malignant, or cancerous cells. The sample may comprise nucleic acids from normal, neoplastic, malignant, or cancerous cells. The sample may be a tumour sample or a sample of a tissue comprising neoplastic or cancerous cells. The sample may be blood or a blood fraction, such as a plasma fraction. The sample may be blood or a blood fraction, such as plasma, comprising circulating tumour DNA or suspected of comprising circulating tumour DNA. The sample may comprise circulating tumour DNA or be suspected of comprising circulating tumour DNA. The sample may be blood or a blood fraction, such as plasma, comprising circulating foetal DNA or suspected of comprising circulating foetal DNA. The sample may comprise circulating foetal DNA or be suspected of comprising circulating foetal DNA. The sample may have been subject to genetic modification or gene editing. For instance, the sample may have been subjected to editing techniques capable of inducing a DSB, for example CRISPR-Cas9, TALEN, or other nucleases. Thus, the library may be generated to allow the detection of off-target mutations induced by an editing technique.

If necessary due to nature of the isolated nucleic acids, the nucleic acids may be sheared or fragmented as a part of step a). Methods of fragmenting nucleic acids are known in the art and may be, for instance, mechanical shearing or enzymatic shearing. The fragmentation may comprise sonication, for instance with a Bioruptor sonicator or a Covaris sonicator. The fragmentation may be enzyme based and may make use of an enzyme-based reagent that shears DNA to produce fragments of desired sizes in a time-dependent manner. Suitable commercially available reagents include NEBNext dsDNA Fragmentase (NEB). The fragmentation may be a performed simultaneously with the first adapter ligation step, for instance via tagmentation. The fragmentation may comprise the use of a nuclease, for instance an endonuclease, endonucleases, a restriction enzyme, or restriction enzymes. The fragmentation may comprise the use of a nucleic acid-guided endonuclease, such as an RNA-guided DNA endonuclease. The fragmentation may comprise the use of Cas protein or a derivative or variant. The fragmentation may comprise the use of Cas9, Cpf1, C2c2, C2c1, CasM, CasMini, a retron, a prokaryotic argonaute, a TALEN, or a meganuclease.

Fragmentation as a part of step a) may not be required for all embodiments. For instance, some nucleic acid sources do not require fragmentation. For example, samples that have been obtained from plasma may not require fragmentation. Alternatively, the nucleic acids may contain double strand breaks (DSBs), which may be naturally occurring or induced, and such samples may not need to be fragmented in step a). In some examples, an adapter may be ligated directly to a DSB. The fragmentation may generate nucleic acid fragments of a particular size or with a particular size distribution, and may be followed by a size selection step. Methods wherein step a) does not comprise fragmentation may also comprise a size selection step. Many systems or reagents for size selection and/or clean-up steps are known in the art. For instance, size selection using beads to remove or select fragments of a certain size. The beads may be Solid Phase Reversible Immobilisation (SPRI) beads. Commercially available beads include “SPRIselect” (Beckman Coulter) or SPRI beads (GC Biotech, CNGS-0005). Capillary DNA electrophoresis may be used for size selection. Capillary DNA electrophoresis may also be used to assess successful ligation and the removal of excess adapters. Other alternatives include gel-based electrophoresis size-selection steps or systems, for instance comprising the use of agarose gels or polyacrylamide gels. Suitable systems are commercially available, such as the BluePippin system (Sage Science). Yet further examples of systems for size selection and/or clean-up include DNA extraction column-based systems.

The method may comprise removing fragments whose size is less than about 100 bp, or less than about 150 bp, and/or retaining fragments whose size is greater than about 150 bp.

In some embodiments, the resultant fragments are 100 to 1500 bp, 200 to 1300 bp, 300 to 1100 bp, 400 to 1000 bp, 500 to 900 bp, or 600 to 800 bp. In a particular embodiment, the nucleic acids are fragments of a size of approximately 600 to 800 bp.

Hence, in an embodiment, step a) may be as follows:
a) providing a plurality of nucleic acids; wherein the providing comprises:
   i) isolating a plurality of nucleic acids from a sample;
   ii) fragmenting said plurality of nucleic acids; and
   iii) selecting the fragments of the plurality of nucleic acids based on size.

The fragmented nucleic acids may be treated to be suitable for adapter ligation. For instance, a binding feature or binding features may be added to the nucleic acids. The binding features may comprise a 5′ feature and/or a 3′ feature. The binding feature may be any suitable for facilitating the ligation of an adapter.

For example, the 5′ or 3′ binding feature may comprise one of the following: a phosphate group; a triphosphate ‘T-tail’, such as a deoxythymidine triphosphate ‘T-tail’; a triphosphate ‘A-tail’, such as a deoxyadenosine triphosphate ‘A-tail’; at least one random N nucleotide, such as a plurality of N nucleotides, or any other known binding group to allow linkage of an adapter to a nucleic acid.

In a particular embodiment, the fragmented nucleic acids are end blunted and A-tailed. Thus, a 5′ phosphate and/or a 3′ A tail may be added to the fragmented nucleic acids.

Hence, in an embodiment, step a) may be as follows:
a) providing a plurality of nucleic acids; wherein the providing comprises:
   i) isolating a plurality of nucleic acids from a sample;
   optionally ii) fragmenting said plurality of nucleic acids;
   optionally iii) selecting the fragments of the plurality of nucleic acids based on size; and
   iv) adding a 5′ and/or a 3′ binding feature to said plurality of nucleic acids.

Steps i), ii), iii), and iv) may be performed in the order i), ii), iii), and then iv). However, any order may be followed that allows the preparation of a plurality of nucleic acids that are suitable for the downstream steps disclosed herein. For instance, the order may be i), ii), iv), and then iii).

In a particular non-limiting embodiment, step a) may be as follows:
a) providing a plurality of nucleic acids; wherein the providing comprises:
   i) isolating a plurality of nucleic acids from a sample, wherein the plurality of nucleic acids is gDNA;
   ii) fragmenting said isolated plurality of nucleic acids;
   iii) selecting the fragments of the plurality of nucleic acids based on size;
   iii) end blunting said selected nucleic acids; and
   iv) adding an A-tail to said end blunted nucleic acids.

In another non-limiting embodiment, step a) may be as follows:
a) providing a plurality of nucleic acids; wherein the providing comprises:
   i) isolating a plurality of nucleic acids from a sample, wherein the plurality of nucleic acids is gDNA;
   ii) fragmenting said isolated plurality of nucleic acids;
   iii) selecting the fragments of the plurality of nucleic acids based on size;
   iii) end blunting and 5′ phosphorylating said selected nucleic acids; and
   iv) adding an A-tail to said end blunted nucleic acids.

In other embodiments, step a) comprises both fragmentation of the nucleic acids and the ligation of an adapter. For instance, a tagmentation step. The ligated adapter may be the non-hairpin adapter or the hairpin adapter, depending on the order in which the steps are performed.

Step a) and step b) may be combined as follows:
i) isolating a plurality of nucleic acids from a sample; and
ii) fragmenting and ligating a non-hairpin adapter to said plurality of nucleic acids; and optionally iii) selecting the fragments of the plurality of nucleic acids based on size.

Alternatively, step a) and step d) may be combined as follows:
i) isolating a plurality of nucleic acids from a sample; and
ii) fragmenting and ligating a hairpin adapter to said plurality of nucleic acids; and
optionally iii) selecting the fragments of the plurality of nucleic acids based on size.

In one embodiment, step a) and step b) are combined as follows:
1) providing a plurality of nucleic acids; and
2) exposing the plurality of nucleic acids to a non-hairpin adapter under conditions conducive to ligation and fragmentation.

In another embodiment, step a) and step d) are combined as follows:
1) providing a plurality of nucleic acids; and
2) exposing the plurality of nucleic acids to a hairpin adapter under conditions conducive to ligation and fragmentation.

In some embodiments, at least one type of adapter is ligated in situ. In this situation, step a) may comprise the permeabilization of a cell or tissue sample. For instance, step a) may comprise exposing a sample to a permeabilizing agent. Nucleic acids, such as DNA or gDNA, may be isolated from the sample after the ligation of an adapter. In these embodiments, the adapter may be ligated to a DSB. The DSB may be naturally occurring or induced.

Step b) comprises exposing the plurality of nucleic acids to a non-hairpin adapter under conditions conducive to ligation. Hence, in some embodiments the non-hairpin adapter will be ligated to the available, or unprotected, ends of the nucleic acids. In embodiments where step b) is performed before step d), this will result in ligation of non-hairpin adapters to both ends of at least a portion of the plurality of nucleic acids. In embodiments where step b) is performed after step d), this will result in ligation of non-hairpin adapters to the end of the nucleic acid at which a hairpin is not present.

As discussed, step b) may be performed separately from or simultaneously with fragmentation. In embodiments where step b) is simultaneous with fragmentation, this may either be the fragmentation of step a) and so as a part of the initial library preparation or, if step b) is performed after step d), then step b) may be combined with step c) (i.e. the fragmentation that takes place after the first adapter ligation step). A “non-hairpin adapter” is an adapter that does not comprise a hairpin loop. For instance, the non-hairpin adapter will not comprise a single nucleic acid strand forming a duplex by virtue of a portion of the single nucleic acid strand hybridising to another portion of the same single nucleic acid strand. A double-stranded non-hairpin adapter may comprise two separate nucleic acid strands, which may form a duplex due to hybridisation between at least a portion of one strand and at least a portion of the other strand.

In some embodiments a non-hairpin adapter is or comprises nucleic acid. In some embodiments, the non-hairpin adapter is or comprises DNA, RNA, and/or xeno nucleic acid (XNA). The non-hairpin adapter may comprise modified and/or un-modified nucleotides. In some embodiments, the non-hairpin adapter is double-stranded. In a particular embodiment, the non-hairpin adapter comprises double-stranded DNA.

The non-hairpin adapter may comprise a sequence that is capable of binding by hybridisation to a primer immobilised to a substrate. For instance, the non-hairpin adapter may comprise a sequence that is at least partially complementary to a primer that is immobilised to a substrate. In some examples, the sequence may be referred to as a site for the hybridisation of a flow cell primer or a bead-bound primer. In such embodiments, the method may be a method of library preparation for nucleic acid sequencing, wherein the preparation comprises modifying nucleic acids to be suitable for binding to a substrate comprising immobilised primers. The length of the complementary region may be 5, 10, 15, 20, 21, 22, 23, 24, or more bases. Alternatively, the complementary region may include 5, 10, 15, 20, 21, 22, 23, 24, or more complementary bases.

The non-hairpin adapter may comprise a sequence that is identical to at least a portion of, or all of, a second primer. The second primer may be immobilised to the substrate or may be in solution. The length of the identical region may be 5, 10, 15, 20, 21, 22, 23, 24, or more bases. The first and the second primer may be configured to allow the amplification of nucleic acids on the substrate.

In particular embodiments, the non-hairpin adapter is ligated as a complete adapter. As such, in these embodiments, no further steps need to be performed in order to add features of the adapter. Thus, the non-hairpin adapter can be ligated to the plurality of nucleic acids as a full adapter without the need for a polymerase step or steps to add or fill in any nucleic acid sequences. In particular, the non-hairpin adapter may be ligated to the plurality of nucleic acids as a molecule that comprises both the sequence that can hybridise to the substrate and the sequence that enables amplification on the substrate.

In a preferred embodiment, the non-hairpin adapter is a Y-adapter. A “Y-adapter” comprises two strands which are only partly complementary, such that the Y-adapter comprises a portion including two non-complementary single strands and a double-stranded complementary portion (e.g. to form a “Y” shape). The terminus of the double-stranded portion may ligate to another nucleic acid and, by virtue of the single-stranded portion, this may result in one sequence being ligated to the 5′ end of a nucleic acid and a different sequence being ligated to the 3′ end of the nucleic acid.

For instance, the Y-adapter may comprise a first nucleic acid (e.g. DNA) strand and a second nucleic acid (e.g. DNA) strand. In an embodiment, the first strand comprises, in the 5′ to 3′ direction, a portion that is complementary to the second strand and a portion that is not complementary to the second strand; and the second strand comprises, in the 5′ to 3′ direction, a portion that is not complementary to the first strand and a portion that is complementary to the first strand.

In particular embodiments, the Y-adapter is ligated as a complete adapter. As such, in these embodiments, no further steps need to be performed in order to add features of the Y-adapter. Thus, the Y-adapter can be ligated to the plurality of nucleic acids as a full adapter without the need for a polymerase step or steps to add or fill in any nucleic acid sequences. In particular, the Y-adapter may be ligated to the plurality of nucleic acids as a molecule that comprises both the sequence that can hybridise to the substrate and the sequence that enables amplification on the substrate.

Y-adapters are known in the art. For instance, the Y-adapter may be an Illumina Y-adapter comprising a P5 binding sequence and a P7 binding sequence. In an embodiment, the Y-adapter comprises the sequence GTGTAGATCTCGGTGGTCGCCGTATCATT (SEQ ID NO: 1) and/or the sequence CAAGCAGAAGACGGCATACGAGAT (SEQ ID NO: 2). In another embodiment, the Y-adapter comprises the sequence ATCTCGTATGCCGTCTTCTGCTTG (SEQ ID NO: 3) and/or AATGATACGGCGACCACCGAGATCTACAC (SEQ ID NO: 4). In an embodiment, the Y-adapter comprises at least 5, 10, 15, 16, 17 18, 19, 20, or all 21 bases of SEQ ID NO: 1. In an embodiment, the Y-adapter comprises at least 5, 10, 15, 16, 17 18, 19, 20, 21, 22, 23, or all 24 bases of SEQ ID NO: 2. In an embodiment, the Y-adapter comprises at least 5, 10, 15, 16, 17 18, 19, 20, 21, 22, 23, or all 24 bases of SEQ ID NO: 3. In an embodiment, the Y-adapter comprises at least 5, 10, 15, 16, 17 18, 19, or all 20 bases of SEQ ID NO: 4. In an embodiment, the Y-adapter comprises at least 5, 10, 15, 16, 17 18, 19, 20, or all 21 bases of SEQ ID NO: 1 and at least 5, 10, 15, 16, 17 18, 19, 20, 21, 22, 23, or all 24 bases of SEQ ID NO: 2. In an embodiment, the Y-adapter comprises at least 5, 10, 15, 16, 17 18, 19, 20, 21, 22, 23, or all 24 bases of SEQ ID NO: 3 and at least 5, 10, 15, 16, 17 18, 19, or all 20 bases of SEQ ID NO: 4. The Y-adapters may comprise sufficient bases of any of SEQ ID NOs: 1 to 4 to allow hybridisation to a complementary primer.

The Y-adapter may comprise a sequence that is capable of binding by hybridisation to a first primer and optionally a sequence that is capable of binding by hybridisation to a second primer. The first and the second primer may be for clonal amplification of the nucleic acid, for instance via bridge amplification. The Y-adapter may comprise a sequence that is capable of binding by hybridisation to a first primer immobilised to a substrate, and a sequence that is identical to at least a portion of, or all of, a second primer immobilised to the substrate. For instance, the Y-adapter may comprise a sequence that is at least partially complementary to a first primer that is immobilised to a substrate. The sequence that is at least partially complementary to a first immobilised primer and the sequence that is identical to at least a portion of a second immobilised primer may be present on different strands of the Y-adapter such that they form at least part of the non-complementary portion of the Y-adapter. In these embodiments, the method may be a method of library preparation for nucleic acid sequencing, wherein the preparation comprises modifying nucleic acids to be suitable for binding to a substrate comprising a first type of immobilised primer and a second type of immobilised primer. In such embodiments, the first immobilised primer and complementary portion of the Y-adapter and the second immobilised primer and identical portion of the Y-adapter may be suitable for performing bridge amplification of the target nucleic acids.

Thus, in an embodiment, the Y-adapter comprises a first strand comprising a sequence that is at least partially complementary to a first primer immobilised to a substrate; and a second strand comprising a sequence that is identical to at least a region of a second primer immobilised to the substrate. In an embodiment, the Y-adapter comprises a first strand comprising at least 5, 10, 15, 16, 17 18, 19, 20, or all 21 bases of SEQ ID NO: 1 and a second strand comprising at least 5, 10, 15, 16, 17 18, 19, 20, 21, 22, 23, or all 24 bases of SEQ ID NO: 2. In an embodiment, the Y-adapter comprises a first strand comprising at least 5, 10, 15, 16, 17 18, 19, 20, 21, 22, 23, or all 24 bases of SEQ ID NO: 3 and a second strand comprising at least 5, 10, 15, 16, 17 18, 19, or all 20 bases of SEQ ID NO: 4. In an embodiment, the Y-adapter comprises a first strand comprising a sequence according to SEQ ID NO: 1 and a second strand comprising a sequence according to SEQ ID NO: 2. In an embodiment, the Y-adapter comprises a first strand comprising a sequence according to SEQ ID NO: 3 and a second strand comprising a sequence according to SEQ ID NO: 4.

In other embodiments, the Y-adapter may comprise a sequence that is capable of binding by hybridisation to a first primer immobilised to a substrate, and a sequence that is identical to at least a portion of a second primer that is not immobilised to the substrate. In an embodiment, the substrate may be a bead.

The non-hairpin adapter may comprise a hybridization site to which a sequencing primer can bind. The non-hairpin adapter may comprise a first hybridisation site to which a first sequencing primer can bind and a second hybridisation site to which a second sequencing primer can bind. The first hybridisation site and the second hybridisation side may be present on different strands of the non-hairpin adapter. The first and second hybridisation sites may be at least partially complementary.

Thus, in an embodiment, the non-hairpin adapter, e.g. Y-adapter, may comprise a first strand comprising a first hybridisation site to which a first sequencing primer can bind; and a second strand comprising a second hybridisation site to which a second sequencing primer can bind.

Examples of suitable hybridisation sites are provided herein as SEQ ID NOs: 5-8. These sequences are purely exemplary. SEQ ID NOs: 5-8 may each comprise from 1 to 7, 1 to 6, 1 to 5, 1 to 4, 1 to 3, 2, or 1 modifications such as substitutions, deletions, or insertions. In an embodiment, the modifications are substitutions. However, the skilled person would appreciate that any modification is acceptable as long as a complementary modification can be made to a cognate primer for sequencing, or as long as the modification does not affect the hybridisation and function of the cognate primer.

In embodiments wherein the non-hairpin adapter comprises both a sequence that is capable of binding by hybridisation to a primer immobilised to a substrate and a hybridisation site to which a sequencing primer can bind, after ligation the adapter may be oriented such that the sequence that is capable of binding by hybridisation to a primer immobilised to a substrate is located nearer to the terminus and the hybridisation site to which a sequencing primer can bind is located nearer to the ligation site.

In a particular embodiment, the non-hairpin adapter is a Y-adapter comprising:
a first strand comprising, in the 5′ to 3′ direction, a first hybridisation site to which a first sequencing primer can bind, and a sequence that is at least partially complementary to a first immobilised primer; and
   a second strand comprising, in the 5′ to 3′ direction, a sequence that is identical to a second immobilised primer and a second hybridisation site to which a second sequencing primer can bind. The first and the second hybridisation site may be at least partially complementary.

The non-hairpin adapter may comprise a 5′ and/or 3′ binding feature or binding features. The binding feature may be any suitable for facilitating the ligation of an adapter. For example, the 5′ or 3′ binding feature may comprise one of the following: a phosphate group; a triphosphate ‘T-tail’, such as a deoxythymidine triphosphate ‘T-tail’; a triphosphate ‘A-tail’, such as a deoxyadenosine triphosphate ‘A-tail’; at least one random N nucleotide, such as a plurality of N nucleotides, or any other known binding group to allow linkage of an adapter to a nucleic acid. In a particular embodiment, the 5′ binding feature is a phosphate group and the 3′ binding feature is a T-tail.

In a particular embodiment, the non-hairpin adapter, e.g. Y-adapter, comprises a first strand comprising a 5′ binding feature, e.g. a phosphate group; and a second strand comprising a 3′ binding feature, e.g. a T-tail.

The non-hairpin adapter may comprise a 5′ and/or 3′ protective feature or protective features, particularly in embodiments where step b) is performed before step d). The protective features may be any that would prevent the ligation of another adapter to the protected adapter. For instance, the protective feature or protective features may prevent the ligation of the hairpin adapter to the non-hairpin adaptor. The non-hairpin adapter may comprise two different terminal protective features. Protective features may not be required for all embodiments, for instance embodiments featuring tagmentation may not require the presence of protective features.

In a particular embodiment, the non-hairpin adapter (e.g. Y-adapter) comprises a first strand comprising a sequence that is at least partially complementary to a first primer immobilised to a substrate and a 3′ protective feature, and a second strand comprising a sequence that is identical to at least a region of a second primer immobilised to the substrate and a 5′ protective feature.

The 5′ and/or 3′ protective features may comprise a feature that provides resistance to any one or more of the following: phosphorylation activity, phosphatase activity, terminal transferase activity, nucleic acid hybridization, endonuclease activity, exonuclease activity, ligase activity, polymerase activity, and protein binding. This can be achieved by any means known to those skilled in the art such as, but not limited to, phosphorothioate linkages, phosphoroamidite spacers, phosphate groups, 2′-O-Methyl groups, inverted deoxy and dideoxy-T modifications, locked nucleic acid bases, dideoxynucleotides, or the like. The protective feature may be a C3 Spacer phosphoramidite (3SpC3). Examples of the activity these features provide are shown in table 1.

In a particular embodiment, the non-hairpin adapter comprises a 5′ inverted ddT and a 3′ C3 Spacer phosphoramidite. In an embodiment, the non-hairpin adapter is a Y-adapter comprising a 5′ inverted ddT and a 3′ C3 Spacer phosphoramidite.

In a particular embodiment, the non-hairpin adapter, e.g. Y-adapter, comprises a first strand comprising a 3′ protective feature, e.g. a C3 Spacer phosphoramidite; and a second strand comprising a 5′ protective feature, e.g. an inverted ddT.

In a particular embodiment, the non-hairpin adapter is a Y-adapter that comprises a first strand comprising, in the 5′ to 3′ direction, a first hybridisation site to which a first sequencing primer can bind, a sequence that is at least partially complementary to a first immobilised primer, and a 3′ protective feature (e.g. a C3 Spacer phosphoramidite); and a second strand comprising, in the 5′ to 3′ direction, a 5′ protective feature (e.g. an inverted ddT), a sequence that is identical to at least a region of a second immobilised primer, and a second hybridisation site to which a second sequencing primer can bind. Optionally the first and second hybridisation sites are at least partially complementary.

In a particular embodiment, the non-hairpin adapter is a Y-adapter that comprises a first strand comprising, in the 5′ to 3′ direction, a 5′ binding feature (e.g. a phosphate group), a first hybridisation site to which a first sequencing primer can bind, a sequence that is at least partially complementary to a first immobilised primer, and a 3′ protective feature (e.g. a C3 Spacer phosphoramidite); and a second strand comprising, in the 5′ to 3′ direction, a 5′ protective feature (e.g. an inverted ddT), a sequence that is identical to at least a region of a second immobilised primer, a second hybridisation site to which a second sequencing primer can bind, and a 3′ binding feature (e.g. a T-tail). Optionally the first and second hybridisation sites are at least partially complementary.

The non-hairpin adapter may optionally comprise an index sequence, which may be referred to as a barcode. The index sequence may allow the identification of sequences from a particular sample. For instance, different samples may be pooled before sequencing and the index may allow the later identification of the sample from which a sequence was derived. This may be referred to as de-multiplexing after sequencing. An index sequence may be positioned such that it is read during sequencing, for instance it may be positioned 3′ to a hybridisation site for a sequencing primer. The index sequence may be a sequence that is at least 3, 4, 5, 6, 7, 8, 9, 10, 11, 12, 13, 14, 15, 20 or more nucleotides long. The index sequence may be a known sequence that is at least 3, 4, 5, 6, 7, 8, 9, 10, 12, 13, 14, 15, 20 or more nucleotides long. The index sequence may be a random sequence that is at least 3, 4, 5, 6, 7, 8, 9, 10, 11, 12, 13, 14, 15, 20 or more nucleotides long. The index sequence may be a degenerate or semi-degenerate sequence. The index sequence may be from 5 to 10 base pairs in length. The index may be 5 or 7 nucleotides long. The index sequence may be present on both strands of a double-stranded portion of an adapter and may be complementary. The non-hairpin adapter may comprise two indexes for dual-indexed sequencing.

The non-hairpin adapter may optionally comprise a Single Molecule Identifier (SMI). Examples of SMIs are disclosed in WO2013/142389, herein incorporated by reference. The SMI may allow the identification of post-amplification nucleic acid molecules that have been derived from a single parent molecule. The SMI sequence may be a double-stranded, complementary SMI sequence or a single-stranded SMI sequence. The SMI sequence may be degenerate or semi-degenerate and may be a random degenerate sequence. A double-stranded SMI sequence may include a first degenerate or semi-degenerate nucleotide n-mer sequence and a second n-mer sequence that is complementary to the first degenerate or semi-degenerate nucleotide n-mer sequence, while a single-stranded SMI sequence may include a first degenerate or semi-degenerate nucleotide n-mer sequence. The first and/or second degenerate or semi-degenerate nucleotide n-mer sequences may be any suitable length to produce a sufficiently large number of unique tags to label a set of sheared DNA fragments from a segment of DNA. Each n-mer sequence may be between approximately 3 to 20 nucleotides in length. Therefore, each n-mer sequence may be approximately 4, 5, 6, 7, 8, 9, 10, 11, 12, 13, 14, 15, 16, 17, 18, 19, 20 nucleotides in length. In one embodiment, the SMI sequence is a random degenerate nucleotide n-mer sequence which is 12 nucleotides in length. With regards to the present invention, it is not essential to include an SMI sequence because no nucleic amplification step is required prior to binding to the substrate. Thus, in some embodiments, the non-hairpin adapter does not comprise an SMI sequence.

The Y-adapter may comprise the sequence GATCGGAAGAGCGTCGTGTAGGGAAAGAGTGT (SEQ ID NO: 5), an index, and SEQ ID NO: 1, and these features may in the recited order from 5′ to 3′. The index may be seven bases long. The Y-adapter may comprise the sequence SEQ ID NO: 2, an index, and the sequence GTGACTGGAGTTCAGACGTGTGCTCTTCCGATCT (SEQ ID NO: 6), and these features may in the recited order from 5′ to 3′. The index may be five bases long.

The Y-adapter may comprise the sequence GATCGGAAGAGCACACGTCTGAACTCCAGTCAC (SEQ ID NO: 7), an index, and SEQ ID NO: 3, and these features may in the recited order from 5′ to 3′. The index may be seven bases long. The Y-adapter may comprise SEQ ID NO: 4, an index, and ACACTCTTTCCCTACACGACGCTCTTCCGATCT (SEQ ID NO: 8), and these features may in the recited order from 5′ to 3′. The index may be five bases long.

In a particular embodiment, the non-hairpin adapter is a Y-adapter comprising:
a first strand comprising, in the 5′ to 3′ direction, SEQ ID NO: 5, optionally an index, and SEQ ID NO: 1; and
a second strand comprising, in the 5′ to 3′ direction, SEQ ID NO: 2, optionally an index, and SEQ ID NO: 6. SEQ ID NOs: 1, 2, 5, and 6 may each comprise from 1 to 7, 1 to 6, 1 to 5, 1 to 4, 1 to 3, 2, or 1 modifications such as substitutions, deletions, or insertions. In an embodiment, the modifications are substitutions.

In a particular embodiment, the non-hairpin adapter is a Y-adapter comprising:
a first strand comprising, in the 5′ to 3′ direction, SEQ ID NO: 7, optionally an index, and SEQ ID NO: 3; and
a second strand comprising, in the 5′ to 3′ direction, SEQ ID NO: 4, optionally an index, and SEQ ID NO: 8. SEQ ID NOs: 3, 4, 7 and 8 may each comprise from 1 to 7, 1 to 6, 1 to 5, 1 to 4, 1 to 3, 2, or 1 modifications such as substitutions, deletions, or insertions. In an embodiment, the modifications are substitutions.

The non-hairpin adapter is provided to the plurality of nucleic acids under conditions conductive to ligation of an adapter to a nucleic acid within the plurality of nucleic acids. The conditions may be varied depending on the nature of the ligation reaction and the binding features of the non-hairpin adapter and the binding features of the plurality of nucleic acids. For instance, the conditions may facilitate the ligation between two double-stranded nucleic acids, wherein each comprise a 5′ phosphate, and wherein one comprises a 3′ A-tail and the other comprises a 3′ T-tail. Other suitable ways of ligating an adapter to a nucleic acid, and the necessary conditions, are known in the art. A purification step may be included after adapter ligation. This step may remove excess adapter molecules. The adapter ligation may be via a technique that also comprises fragmentation, for instance tagmentation.

In embodiments where step b) is performed before step d), the ligation reaction results in the ligation of the non-hairpin adapter to both ends of at least a portion of the plurality of nucleic acids. This may be referred to as a first library. In embodiments where the non-hairpin adapter is a Y-adapter, the first library comprises fragments of nucleic acids to be sequenced, wherein a Y-adapter is ligated to each end of at least a portion of the fragments. Hence, a first nucleic acid sequence may be ligated to the 5′ ends of the strands within the fragments and a second nucleic acid sequence may be ligated to the 3′ ends of the strands within the fragments.

In embodiments where step b) is performed after step d), the ligation reaction results in ligation of a non-hairpin adapter to the end of the nucleic acid at which a hairpin is not present. Hence, at least a portion of the nucleic acids to be sequenced comprise a hairpin at one end and a non-hairpin adapter at the other end. This may be referred to as a second library. In embodiments where the non-hairpin adapter is a Y-adapter, the second library comprises fragments of nucleic acids to be sequenced, wherein at least a portion of the fragments comprise a Y-adapter ligated to one end and a hairpin at the other end.

Step c) comprises fragmenting the plurality of nucleic acids, and may follow either step b) or step d). Step c) is applied to the first library and is performed before or during the formation of the second library.

Methods of fragmenting nucleic acids are known in the art and may be, for instance, mechanical shearing or enzymatic shearing. The fragmentation may comprise sonication, for instance with a Bioruptor sonicator or a Covaris sonicator. The fragmentation may be enzyme based and may make use of an enzyme-based reagent that shears DNA to produce fragments of desired sizes in a time-dependent manner. Suitable commercially available reagents include NEBNext dsDNA Fragmentase (NEB). The fragmentation may be a performed simultaneously with the second adapter ligation step, for instance via tagmentation. The fragmentation may lead to double-strand breaks in the plurality of nucleic acids. The fragmentation might not be site specific and so may induce random breaks, such as random double-strand breaks. The fragmentation leads to double-strand breaks to which an adapter can be ligated, optionally after end repair or similar steps. The fragmentation may lead to double-strand breaks to which an adapter can be ligated without the need for prior polymerase-based steps that make use of one strand as a template. In some embodiments, the fragmentation does not comprise the use of a site-specific nickase. In some embodiments, the fragmentation does not comprise the use of a site-specific nickase to result in a single-stranded portion, which is then repaired using a template-based polymerase.

The fragmentation may comprise the use of a nuclease, for instance an endonuclease, endonucleases, a restriction enzyme, or restriction enzymes. The fragmentation may comprise the use of a nucleic acid-guided endonuclease, such as an RNA-guided DNA endonuclease. The fragmentation may comprise the use of Cas protein or a derivative or variant. The fragmentation may comprise the use of Cas9, Cpf1, C2c2, C2c1, CasM, CasMini, a retron, a prokaryotic argonaute, a TALEN, or a meganuclease.

The fragmentation may generate nucleic acid fragments of a particular size or with a particular size distribution, and may be followed by a size selection step. Many systems or reagents for size selection and/or clean-up steps are known in the art. For instance, size selection using beads to remove or select fragments of a certain size. The beads may be Solid Phase Reversible Immobilisation (SPRI) beads. Commercially available beads include “SPRIselect” (Beckman Coulter) or SPRI beads (GC Biotech, CNGS-0005). Capillary DNA electrophoresis may be used for size selection. Capillary DNA electrophoresis may also be used to assess successful ligation and the removal of excess adapters. Other alternatives include gel-based electrophoresis size-selection steps or systems, for instance comprising the use of agarose gels or polyacrylamide gels. Suitable systems are commercially available, such as the BluePippin system (Sage Science). Yet further examples of systems for size selection and/or clean-up include DNA extraction column-based systems.

If the sample was fragmented before or during the ligation of the first adapter, step c) may comprise selecting for fragments that are approximately half the size of the preceding fragmentation step.

Step c) may comprise removing fragments whose size is less than about 100 bp, or less than about 150 bp, and/or retaining fragments whose size is greater than about 150 bp.

In some embodiments, the resultant fragments are 100 to 700 bp, 150 to 650 bp, 200 to 600 bp, 250 to 550 bp, 300 to 500 bp, or 350 to 450 bp. In some embodiments, the resultant fragments are 150 to 600 bp, 200 to 550 bp, 250 to 500 bp, 275 to 450 bp, or 300 to 400 bp.

The fragmented nucleic acids may be treated to be suitable for adapter ligation. For instance, a binding feature or binding features may be added to the nucleic acids. The binding features may comprise a 5′ feature and/or a 3′ feature. The binding feature may be any suitable for facilitating the ligation of an adapter, including any binding feature disclosed herein. If step b) was performed to generate the first library, the binding features may not be added to the non-hairpin adapter due to the presence of the protective features on the non-hairpin adapter.

In a particular embodiment, the fragmented nucleic acids are end blunted and A-tailed. Thus, a 5′ phosphate and/or a 3′ A-tail may be added to the fragmented nucleic acids.

Hence, in an embodiment, step c) may be as follows:
c) fragmenting the plurality of nucleic acids; and further comprising:
   optionally i) selecting the fragments of the plurality of nucleic acids based on size; and
ii) adding a 5′ and/or a 3′ binding feature to said plurality of nucleic acids.

Steps i) and ii) may be performed in the order i) and then ii). However, any order may be followed that allows the preparation of a plurality of nucleic acids that are suitable for the downstream steps disclosed herein.

In a particular non-limiting embodiment, step c) may be as follows:
c) fragmenting the plurality of nucleic acids; and further comprising:
   i) selecting the fragments of the plurality of nucleic acids based on size;
   ii) end blunting said selected nucleic acids; and
   iii) adding an A-tail to said end blunted nucleic acids.

In other embodiments, step c) comprises a step that both fragments the nucleic acids and ligates an adapter. For instance, a tagmentation step. The ligated adapter may be the non-hairpin adapter or the hairpin adapter, depending on the order in which the steps are performed.

Thus, step c) and step b) may be combined as follows:
i) fragmenting and ligating a non-hairpin adapter to said plurality of nucleic acids; and
optionally ii) selecting the fragments of the plurality of nucleic acids based on size.

Alternatively, step c) and step d) may be combined as follows:
i) fragmenting and ligating a hairpin adapter to said plurality of nucleic acids; and optionally ii) selecting the fragments of the plurality of nucleic acids based on size.

In other embodiments, step c) may comprise a tagmentation step that inserts a recognition site into the fragmented nucleic acids. For instance, a recognition site for an enzyme capable of forming a hairpin, such as protelomerase. The protelomerase may be TelN.

In some examples, step d) comprises exposing the plurality of nucleic acids to a hairpin adapter under conditions conducive to ligation. Hence, the hairpin adapter will be ligated to the available, or unprotected, ends of the nucleic acids. In embodiments where step d) is performed before step b), this will result in ligation of hairpin adapters to both ends of at least a portion of the plurality of nucleic acids. In embodiments where step d) is performed after step b), this will result in ligation of non-hairpin adapters to the end of the nucleic acid to which a hairpin adapter is not ligated.

In other examples, step d) comprises exposing the plurality of nucleic acids to conditions capable of capable of forming a hairpin at an end of a nucleic acid molecule. For instance, step d) may comprise the use of conditions or an enzyme capable of generating covalently closed ends in double stranded nucleic acid molecule. An example of a suitable enzyme is a protelomerase, such as TelN. A TelN recognition sequence may be present in, or may have been introduced into, the plurality of nucleic acids. For instance, a TelN recognition sequence may be introduced as part of a fragmentation via tagmentation.

As discussed, step d) may be performed separately from or simultaneously with fragmentation. In embodiments where step d) is simultaneous with fragmentation, this may either be the fragmentation of step a) and so as a part of the initial library preparation or, if step d) is performed after step b), then step d) may be combined with step c) (i.e. the fragmentation that takes place after the first adapter ligation step).

A “hairpin” adapter comprises a hairpin loop. Hairpin adapters can comprise a single nucleic acid strand forming a duplex by virtue of a portion of the single nucleic acid strand hybridising to another portion of the same single nucleic acid strand. Hairpin adapters are known in the art. A hairpin adapter may be referred to as a “U-adapter”.

A double-stranded nucleic acid that has a hairpin present at only one end is capable of being denatured to form a unitary single-stranded molecule including both strands of the original double-stranded nucleic acid.

In some embodiments a hairpin adapter is or comprises nucleic acid. In some embodiments, the hairpin adapter is or comprises DNA, RNA, and/or XNA. The hairpin adapter may comprise modified and/or un-modified nucleotides. In a particular embodiment, the hairpin adapter comprises DNA.

A non-limiting example of a hairpin adapter is: GGGCCTADDDDDDDDTAGGCCCT (SEQ ID NO: 9), where D is G, A or T (but not C).

The hairpin adapter may be provided to the plurality of nucleic acids under conditions conductive to ligation of an adapter to a nucleic acid within the plurality of nucleic acids. The conditions may be varied depending on the nature of the ligation reaction and the binding features of the hairpin adapter and the binding features of the plurality of nucleic acids. For instance, the conditions may facilitate the ligation between two double-stranded nucleic acids, wherein each comprise a 5′ phosphate, and wherein one comprises a 3′ A-tail and the other comprises a 3′ T-tail. Other suitable ways of ligating an adapter to a nucleic acid, and the necessary conditions, are known in the art. A purification step may be included after adapter ligation. This step may remove excess adapter molecules.

In embodiments where step d) is performed before step b), step d) results in the ligation of the hairpin adapter to both ends of at least a portion of the plurality of nucleic acids, or the formation of a hairpin at both ends of at least a portion of the plurality of nucleic acids. This may be referred to as a first library. In such embodiments, the method may comprise a step of removing any linear nucleic acids. This step may result in only the nucleic acids with a hairpin present at both ends, which are essentially nucleic acid circles, being retained.

In embodiments where step d) is performed after step b), step d) results in ligation of a hairpin adapter to the end of the nucleic acid to which a non-hairpin adapter is not ligated, or the formation of a hairpin at the end of the nucleic acid to which a non-hairpin adapter is not ligated. Hence, at least a portion of the nucleic acids to be sequenced comprise a hairpin at one end and a non-hairpin adapter at the other end. This may be referred to as a second library.

As discussed, step d) may be performed separately from or simultaneously with fragmentation. In embodiments where step d) is simultaneous with fragmentation, this may either be the fragmentation of step a) and so as a part of the initial library preparation or, if step d) is performed after step b), then step d) may be combined with step c) (i.e. the fragmentation that takes place after the first adapter ligation step).

In an embodiment, there is provided a method of library preparation for nucleic acid sequencing, the method comprising the following sequential steps in the recited order:
providing a plurality of nucleic acids;
exposing the plurality of nucleic acids to a non-hairpin adapter under conditions conducive to ligation;
fragmenting the plurality of nucleic acids; and
exposing the plurality of nucleic acids to a hairpin adapter under conditions conducive to ligation.

In an embodiment, there is provided a method of library preparation for nucleic acid sequencing, the method comprising the following sequential steps in the recited order:
providing a plurality of nucleic acids;
exposing the plurality of nucleic acids to a non-hairpin adapter under conditions conducive to ligation;
fragmenting the plurality of nucleic acids; and
exposing the plurality of nucleic acids to conditions capable of forming a hairpin at an end of a nucleic acid molecule.

In another embodiment, there is provided a method of library preparation for nucleic acid sequencing, the method comprising the following sequential steps in the recited order:
providing a plurality of nucleic acids;
exposing the plurality of nucleic acids to a non-hairpin adapter under conditions conducive to ligation; and
exposing the plurality of nucleic acids to a hairpin adapter under conditions conducive to ligation and fragmentation.

In an embodiment, there is provided a method of library preparation for nucleic acid sequencing, the method comprising the following sequential steps in the recited order:
providing a plurality of nucleic acids;
exposing the plurality of nucleic acids to a hairpin adapter under conditions conducive to ligation;
fragmenting the plurality of nucleic acids; and
exposing the plurality of nucleic acids to a non-hairpin adapter under conditions conducive to ligation.

In an embodiment, there is provided a method of library preparation for nucleic acid sequencing, the method comprising the following sequential steps in the recited order:
providing a plurality of nucleic acids;
exposing the plurality of nucleic acids to conditions capable of forming a hairpin at an end of a nucleic acid molecule;
fragmenting the plurality of nucleic acids; and
exposing the plurality of nucleic acids to a non-hairpin adapter under conditions conducive to ligation.

In another embodiment, there is provided a method of library preparation for nucleic acid sequencing, the method comprising the following sequential steps in the recited order:
providing a plurality of nucleic acids;
exposing the plurality of nucleic acids to a hairpin adapter under conditions conducive to ligation; and
exposing the plurality of nucleic acids to a non-hairpin adapter under conditions conducive to ligation and fragmentation.

In some embodiments, the method may further comprise contacting the plurality of nucleic acids, which may be referred to as a second library at this stage, to a substrate comprising immobilised primers, under conditions suitable for the hybridisation of a portion of the non-hairpin adapter at least a portion of an immobilised primer.

Any nucleic acids lacking a ligated non-hairpin adapter will not hybridise to the flow cell. For instance, any nucleic acids that include a hairpin adapter at both ends.

The substrate may be a solid surface such as a surface of a flow cell, a bead, a slide, or a membrane. In particular, the substrate may be a flow cell. The substrate may be a patterned or a non-patterned flow cell. The substrate may comprise glass, quartz, silica, metal, ceramic, or plastic. The substrate surface may comprise a polyacrylamide matrix or coating.

As used herein, the term “flow cell” is intended to have the ordinary meaning in the art, in particular in the field of sequencing by synthesis. Exemplary flow cells include, but are not limited to, those used in a nucleic acid sequencing apparatus such as flow cells for the Genome Analyzer®, MiSeq®, NextSeq®, HiSeq®, or NovaSeq® platforms commercialised by Illumina, Inc. (San Diego, Calif.); or for the SOLiD™ or Ion Torrent™ sequencing platform commercialized by Life Technologies (Carlsbad, Calif.). Exemplary flow cells and methods for their manufacture and use are also described, for example, in WO2014/142841A1; U.S. Pat. App. Pub, No. 2010/0111768 A1 and U.S. Pat. No. 8,951,781.

The substrate may comprise immobilised primers, for instance two types of primer which together can act as forward and reverse primers for bridge amplification. Immobilisation to a substrate means that the primer is bound to the substrate even under conditions that would denature double-stranded nucleic acids. For instance, the primer may be covalently bound to the substrate. The primers are oriented such that the 5′ end is proximal and the 3′ end is distal to the point of immobilisation. Such arrangements are standard in the art.

In a particular embodiment, the substrate may comprise a first and a second immobilised primer. The immobilised primers may, in some embodiments, be suitable for acting as primers during bridge amplification. Bridge amplification may result in clonal amplification of nucleic acids immobilised to a substrate. The non-hairpin adapter may be a Y-adapter comprising a sequence that is complementary to the first immobilised primer and a sequence that is identical to the second immobilised primer. Thus, in such embodiments the second library comprises nucleic acids that include a hairpin adapter at one end and, at the other end, a sequence complementary to the first immobilised primer ligated to one strand and a sequence that is identical to the second immobilised primer ligated to the other strand. The sequence complementary to the first immobilised primer may be ligated to the 3′ end of the nucleic acid and the sequence that is identical to the second immobilised primer may be ligated to the 5′ end of the nucleic acid.

In some embodiments, the second library may be denatured before being contacted to the substrate, such that the nucleic acids of the second library are single stranded. In some embodiments, the second library may be contacted to the substrate under denaturing conditions such that nucleic acids within the library are single-stranded at the time of contact.

In a particular embodiment, there is disclosed a method of library preparation for nucleic acid sequencing, wherein the preparation comprises modifying nucleic acids to be suitable for binding to a substrate comprising a first immobilised primer, the method comprising:
a) providing a plurality of nucleic acids;
b) exposing the plurality of nucleic acids to a non-hairpin adapter under conditions conducive to ligation, wherein the non-hairpin adapter comprises a sequence complementary to the first immobilised primer;
c) fragmenting the plurality of nucleic acids; and
d) exposing the plurality of nucleic acids to a hairpin adapter under conditions conducive to ligation, or exposing the plurality of nucleic acids to conditions capable of forming a hairpin at an end of a nucleic acid molecule. In one embodiment, the steps may be performed in the order a), b), c), and then d). These steps may be sequential or steps c) and d) may be combined, for instance as a tagmentation step. Step b) may be combined with an earlier fragmentation step. In another embodiment, the steps may be performed in the order a), d), c), and then b). These steps may be sequential or steps c) and b) may be combined, for instance as a tagmentation step. Step d) may be combined with an earlier fragmentation step.

Following the above-mentioned steps, the method may further comprise:
e) contacting the plurality of nucleic acids to the substrate under conditions suitable for hybridisation of the first immobilised primer to complementary nucleic acids. As an example, the substrate may be a flow cell suitable for nucleic acid sequencing.

In particular embodiments, no nucleic acid amplification step, such as PCR, is performed before step e). For instance, the method may be performed starting with a tissue sample and ending with fragments of the gDNA from the sample bound to a sequencing flow cell via ligated adapters that are hybridised to immobilised primers; wherein no nucleic acid amplification step, such as a PCR step, was performed during this process. While a PCR step could be included in order to amplify targets, the inventors have surprising found that this is not a requirement of the methods of the invention. The exclusion of an amplification step may advantageously avoid the introduction of bias or the introduction of sequence errors as a result of the amplification. Thus, methods of the present invention that exclude an amplification step may be used for whole-genome error-corrected sequencing.

Hence, in an embodiment, there is disclosed a method of library preparation for nucleic acid sequencing, wherein the preparation comprises modifying nucleic acids to be suitable for binding to a substrate comprising a first immobilised primer, the method comprising the following steps in the recited order, wherein steps c) and d) may be combined:
a) providing a plurality of nucleic acids;
b) exposing the plurality of nucleic acids to a non-hairpin adapter under conditions conducive to ligation, wherein the non-hairpin adapter comprises a sequence complementary to the first immobilised primer;
c) fragmenting the plurality of nucleic acids;
d) exposing the plurality of nucleic acids to a hairpin adapter under conditions conducive to ligation, or exposing the plurality of nucleic acids to conditions capable of forming a hairpin at an end of a nucleic acid molecule; and
e) contacting the plurality of nucleic acids to the substrate under conditions suitable for hybridisation of the first immobilised primer to complementary nucleic acids;
   wherein no nucleic acid amplification step, e.g. PCR, is performed before step e). The non-hairpin adapter may be any disclosed herein, such as a Y-adapter. The substrate may be any disclosed herein, such as a flow cell.

In an alternative embodiment, there is disclosed a method of library preparation for nucleic acid sequencing, wherein the preparation comprises modifying nucleic acids to be suitable for binding to a substrate comprising a first immobilised primer, the method comprising the following steps in the recited order, wherein steps c) and d) may be combined:
a) providing a plurality of nucleic acids;
b) exposing the plurality of nucleic acids to a hairpin adapter under conditions conducive to ligation, or exposing the plurality of nucleic acids to conditions capable of forming a hairpin at an end of a nucleic acid molecule;
c) fragmenting the plurality of nucleic acids;
d) exposing the plurality of nucleic acids to a non-hairpin adapter under conditions conducive to ligation, wherein the non-hairpin adapter comprises a sequence complementary to the first immobilised primer; and
e) contacting the plurality of nucleic acids to the substrate under conditions suitable for hybridisation of the first immobilised primer to complementary nucleic acids;
   wherein no nucleic acid amplification step, e.g. PCR, is performed before step e). The non-hairpin adapter may be any disclosed herein, such as a Y-adapter. The substrate may be any disclosed herein, such as a flow cell.

After step e), the methods may further comprise contacting any hybridised nucleic acid with a polymerase under conditions suitable for the extension of the immobilised primer to synthesise a nucleic acid which is a chain of nucleotides that are complementary to the hybridised nucleic acid. The newly formed nucleic acid may then be amplified. In some embodiments, the primer for amplification is also immobilised to the substrate and may, for instance, be suitable for bridge amplification. This process is known in the art and forms clonal clusters of nucleic acids. In other examples, the primer for amplification may be in solution, for instance for embodiments wherein the substrate is a bead. The amplified nucleic acids may then be sequenced in the usual way, for instance by sequencing-by-synthesis. The non-hairpin adaptor may comprise a site for the binding of a sequencing primer to assist this process. The non-hairpin adaptor may also comprise an index.

Thus, in an embodiment, the methods may further comprise:
f) obtaining sequence information for any nucleic acids that hybridised to the substrate in step e).

In embodiments where step e) is not carried out, sequence information may be obtained by sequencing the second library.

Methods including a step of obtaining sequence information may be referred to as a method for nucleic acid sequencing or as a method for error-corrected nucleic acid sequencing. Such methods are “error-corrected” because sequence information is derived from both strands of a portion of a double-stranded nucleic acid and hence any errors that have been introduced after provision of the nucleic acids for sequencing may be corrected by comparing the sequence obtained for one strand to the sequence obtained for the other strand. In essence, each portion of the original nucleic acid sample is read twice, and each read is of an independent sequence, hence allowing error correction of any discrepancies that are only present in a single read.

In an embodiment, the method is for the identification of mutations, and the method includes identifying as mutations any changes in the expected sequence that are consistent on both strands of a DNA molecule, and not identifying any changes in the expected sequence as a mutation if the change is not consistent on both strands of the DNA molecule. Such methods may include the bioinformatic alignment of the sequence reads to a reference sequence, in order to identify deviations from the expected sequence. The reference sequence may be a known sequence for example the human genome, such as the human genome reference sequence Human Build 38 patch release 14 (GRCh38.p14; Genome Reference Consortium) in the NCBI database.

In particular embodiments, the methods may be applied to gDNA obtained from a sample and may be for unbiased genome-wide error-corrected sequencing. In some embodiments, the methods may be employed to detect off-target effects of gene editing techniques. For instance, the methods may be used to detect off-target effects of CRISPR-Cas9 editing, TALEN editing, or any other method of altering the sequence of a nucleic acid.

Methods of sequencing nucleic acids, such as immobilised nucleic acid clusters, are known in the art. In some embodiments, the sequencing may involve the use of a sequencing primer or sequencing primers. For instance, embodiments of the non-hairpin adapter described herein may comprise a first hybridisation site to which a first sequencing primer can bind, and step f) may comprise the use of the first sequencing primer. In some embodiments, the non-hairpin adapter described herein may also comprise a second hybridisation site to which a second sequencing primer can bind, and step f) may also comprise the use of the second sequencing primer.

The sequencing may be next-generation sequencing or may be massively parallel sequencing.

In a particular embodiment, there is provided a method of library preparation for nucleic acid sequencing wherein the preparation comprises modifying nucleic acids to be suitable for binding to a substrate comprising immobilised primers, the method comprising:
a) providing a plurality of nucleic acids;
b) exposing the plurality of nucleic acids to a Y-adapter under conditions conducive to ligation to generate a first library; wherein the Y-adapter comprises:
   a first strand comprising a sequence that is at least partially complementary to a first primer immobilised to a substrate and optionally a 3′ protective feature, and
   a second strand comprising a sequence that is identical to at least a region of a second primer immobilised to the substrate and optionally a 5′ protective feature;
c) fragmenting the first library, and further comprising:
   i) selecting the fragments of the plurality of nucleic acids based on size; and
d) exposing the selected fragments to a hairpin adapter under conditions conducive to ligation to generate a second library, or exposing the plurality of nucleic acids to conditions capable of forming a hairpin at an end of a nucleic acid molecule.

In another embodiment, there is provided a method of library preparation for nucleic acid sequencing wherein the preparation comprises modifying nucleic acids to be suitable for binding to a substrate comprising immobilised primers, the method comprising:
a) providing a plurality of nucleic acids;
b) exposing the plurality of nucleic acids to a Y-adapter under conditions conducive to ligation to generate a first library; wherein the Y-adapter comprises:
   a first strand comprising a sequence that is at least partially complementary to a first primer immobilised to a substrate and optionally a 3′ protective feature, and
   a second strand comprising a sequence that is identical to at least a region of a second primer immobilised to the substrate and optionally a 5′ protective feature; and
(combined steps) c) and d) exposing the first library to a hairpin adapter under conditions conducive to ligation and fragmentation to generate a second library; optionally wherein tagmentation is performed.

The above two embodiments may be methods of obtaining sequence information from nucleic acids, where the method further comprises:
e) denaturing the second library to produce single-stranded nucleic acids and contacting the single-stranded nucleic acids to the substrate under conditions suitable for hybridisation of the first immobilised primer to complementary nucleic acids, and optionally generating clusters of immobilised nucleic acids via bridge amplification, wherein the first and second immobilised primers act as primers for bridge amplification; and
f) obtaining sequence information for any nucleic acids that hybridised to the substrate in step e).

In a second aspect, there is provided a nucleic acid library obtained or obtainable by any method of the first aspect of the present disclosure. The library of the second aspect is referred to as the second library with regards to the first aspect of the present disclosure.

The nucleic acid library of the second aspect comprises nucleic acids for which sequence information is desired, which may be referred to as target nucleic acids and may be DNA derived from a sample (or derived from said DNA). The DNA may be derived from a mammalian or human sample. The target nucleic acids may be fragments of gDNA or may be derived from said gDNA.

The library comprises a portion of target nucleic acids that have a ligated non-hairpin adapter, as disclosed herein, at one end and a ligated hairpin adapter at the other end. The non-hairpin adapter ligated to the nucleic acids of the library of the invention may be any as disclosed herein. In an embodiment, a portion of the target nucleic acids has a ligated Y-adapter, as disclosed herein, at one end and a ligated hairpin adapter, as disclosed herein, at the other end. The Y-adapter may be an Illumina Y-adapter comprising a P5 binding sequence and a P7 binding sequence.

The present disclosure encompasses libraries of the second aspect that have been denatured to form single strands, such that the portion that formed a hairpin forms a linker between the two strands of the target nucleic acid, and the non-hairpin adapter is present as a sequence at the 5′ terminus and a sequence at the 3′ terminus.

In addition, to the species described above, the nucleic acid library of the second aspect may comprise target nucleic acids that have a hairpin at both ends. Such species will not bind to the substrate and so are not sequencable.

Compared to prior art techniques, the nucleic acid library of the second aspect comprises a reduced amount of target nucleic acids that have a non-hairpin adapter ligated to both ends. In some embodiments, the nucleic acid library does not comprise, or does not comprise a substantial amount of, target nucleic acids that have a non-hairpin adapter ligated to both ends. Such species are sequencable but not error correctable, and so the reduction or avoidance of this species allows for improved sequencing accuracy. In addition, the reduction of this species can allow for the library to be sequenced without a prior amplification step, for instance it can allow the library to be sequenced on a substrate without amplification prior to the application to the substrate.

In examples, the library may comprise less than 99.9%, 99%, 95%, 90%, 80%, 70%, 60%, 50%, 40%, 30%, 20%, 10%, 5%, 3%, 1%, 0.1%, or 0.01% by mass target nucleic acids that have a non-hairpin adapter ligated to both ends.

Thus, in a particular embodiment, there is disclosed a nucleic acid library comprising a target nucleic acid with a non-hairpin adapter ligated to one end and a hairpin at the other end. The nucleic acid library does not comprise, comprises a reduced amount of, or does not comprise a substantial amount of a target nucleic acid with a non-hairpin adapter ligated to one end and a non-hairpin adapter ligated to the other end. The reduction may be in comparison to a library prepared in the same manner but where the first and second adapter ligation steps are performed simultaneously.

In particular, the nucleic acid library of the second aspect may be suitable for methods of sequencing that involve contacting the library with a substrate to bind a portion of the library to the substrate. The non-hairpin adapter may comprise a sequence that is at least partially complementary to a first primer that is immobilised to the substrate. Thus, in an embodiment, there is disclosed a nucleic acid library suitable for methods of sequencing that involve contacting the library to a substrate to bind a portion of the library to the substrate, comprising: i) a target nucleic acid with a non-hairpin adapter ligated to one end and a hairpin at the other end, wherein the non-hairpin adapter comprises a sequence that is at least partially complementary to a first primer that is immobilised to the substrate; and optionally ii) a target nucleic acid with a hairpin at one end and a hairpin at the other end.

In a third aspect, there is disclosed a method of sequencing, wherein the method comprises obtaining sequence information for nucleic acids within a library of the second aspect of the present disclosure.

In an embodiment, there is disclosed a method of obtaining sequencing information, wherein the method comprises:
1) contacting a library of the second aspect of the present disclosure to a substrate comprising a first immobilised primer under conditions suitable for hybridisation of the first immobilised primer to complementary nucleic acids; and
2) obtaining sequence information for any nucleic acids that hybridised to the substrate in step 1).

Step 1) of the third aspect has the same features as step e) of the first aspect of the present disclosure. Step 2) of the third aspect has the same features as step f) of the first aspect of the present disclosure.

In an embodiment, there is disclosed a method of obtaining sequencing information, wherein the method comprises:
1) contacting a nucleic acid library to a substrate comprising a first immobilised primer under conditions suitable for hybridisation of the first immobilised primer to complementary nucleic acids; and
2) obtaining sequence information for any nucleic acids that hybridised to the substrate in step 1);
   wherein the nucleic acid library has been prepared or is obtainable by a method comprising:
a) providing a plurality of nucleic acids;
b) exposing the plurality of nucleic acids to a non-hairpin adapter under conditions conducive to ligation;
c) fragmenting the plurality of nucleic acids; and
d) exposing the plurality of nucleic acids to a hairpin adapter under conditions conducive to ligation, or exposing the plurality of nucleic acids to conditions capable of forming a hairpin at an end of a nucleic acid molecule;
wherein steps b) and d) are performed separately.

As disclosed herein, the inventors have surprisingly discovered that found that amplification of the nucleic acids from the sample, prior to binding to the substrate, is not a requirement of the methods of the invention.

Thus, in a fourth aspect, there is provided a method of library preparation for nucleic acid sequencing, the method comprising:
i) providing a plurality of nucleic acids;
ii) exposing the plurality of nucleic acids to a non-hairpin adapter under conditions conducive to ligation; and
iii) exposing the plurality of nucleic acids to a hairpin adapter under conditions conducive to ligation, or exposing the plurality of nucleic acids to conditions capable of forming a hairpin at an end of a nucleic acid molecule;
   wherein the nucleic acids are not amplified during preparation of the library.

The features disclosed in connection with step a) of the first aspect of the present disclosure are also applicable to step i) of the fourth aspect.

The non-hairpin adapter of the fourth aspect may be any as disclosed for the first aspect of the present disclosure. The non-hairpin adapter may include protective features and/or binding features as disclosed in relation to the first aspect. The hairpin adapter of the fourth aspect may be any as disclosed for the first aspect of the present disclosure. The conditions capable of forming a hairpin at an end of a nucleic acid molecule may be any as disclosed for the first aspect of the present disclosure.

The nucleic acids are not amplified during preparation of the library according to the fourth aspect. For instance, no PCR step is performed.

In a particular embodiment, the steps of the fourth aspect are performed in the order i), ii), and then iii). In another embodiment, the steps of the fourth aspect are performed in the order i), iii), and then ii). In a particular embodiment, steps ii) and iii) are performed separately and a fragmentation step is included between the steps. In another embodiment, the second ligation step may comprise fragmentation, for instance it may be a tagmentation step. The features disclosed in connection with step c) of the first aspect of the present disclosure are also applicable to the fragmenting step of the fourth aspect.

The nucleic acid library generated by steps i), ii), and iii) may be referred to as a second library. Sequence information may be obtained from the second library. In an embodiment, the non-hairpin adapter comprises a sequence that is at least partially complementary to a first primer that is immobilised to a substrate, and the method comprises step iv), contacting the second library to a substrate comprising a first immobilised primer under conditions suitable for hybridisation of the first immobilised primer to complementary nucleic acids. The features disclosed in connection with step e) of the first aspect of the present disclosure are also applicable to step iv) of the fourth aspect. The features disclosed in connection with obtaining sequence information for the first aspect are also applicable to the fourth aspect. In these embodiments, no nucleic acid amplification step is performed prior to step iv).

In a fifth aspect, there is provided a nucleic acid library obtained or obtainable by any method of the fourth aspect of the present disclosure. The library of the fifth aspect is referred to as the second library with regards to the first aspect of the present disclosure.

The library of the fifth aspect does not comprise target nucleic acids that have been amplified, for instance the target nucleic acids have not been subjected to a PCR reaction. The remaining features of the library of the fifth aspect may be as disclosed for the second aspect of the present disclosure.

In a sixth aspect, there is disclosed a method of sequencing, wherein the method comprises obtaining sequence information for nucleic acids within a library of the fifth aspect of the present disclosure.

In an embodiment, there is disclosed a method of obtaining sequencing information, wherein the method comprises:
1) contacting a library of the fifth aspect of the invention to a substrate comprising a first immobilised primer under conditions suitable for hybridisation of the first immobilised primer to complementary nucleic acids; and
2) obtaining sequence information for any nucleic acids that hybridised to the substrate in step 1).

Step 1) of the sixth aspect has the same features as step e) of the first aspect of the present disclosure. Step 2) of the sixth aspect has the same features as step f) of the first aspect of the present disclosure.

In a seventh aspect, there is provided a non-hairpin adapter comprising: a first strand comprising, in the 5′ to 3′ direction, a sequence that is at least partially complementary to a first immobilised primer, and a 3′ protective feature; and a second strand comprising, in the 5′ to 3′ direction, a 5′ protective feature, and a sequence that is identical to at least a region of a second primer.

The non-hairpin adapter of the seventh aspect may comprise any sequence that is at least partially complementary to a first immobilised primer as disclosed for the first aspect of the present disclosure. The non-hairpin adapter of the seventh aspect may comprise any sequence that is identical to at least a region of a second primer as disclosed for the first aspect of the present disclosure. The sequence that is at least partially complementary to a first immobilised primer may be SEQ ID NO: 1 or SEQ ID NO: 3. The sequence that is identical to at least a region of a second immobilised primer may be SEQ ID NO: 2 or SEQ ID NO: 4. In an embodiment, the non-hairpin adapter comprises at least 5, 10, 15, 16, 17 18, 19, 20, or all 21 bases of SEQ ID NO: 1 and/or comprises at least 5, 10, 15, 16, 17 18, 19, 20, 21, 22, 23, or all 24 bases of SEQ ID NO: 2. In an embodiment, the non-hairpin adapter comprises at least 5, 10, 15, 16, 17 18, 19, 20, 21, 22, 23, or all 24 bases of SEQ ID NO: 3 and/or comprises at least 5, 10, 15, 16, 17 18, 19, or all 20 bases of SEQ ID NO: 4. The non-hairpin adapter may comprise sufficient bases of any of SEQ ID NOs: 1 to 4 to allow hybridisation to a complementary primer.

In a particular embodiment, the non-hairpin adapter is a Y-adapter comprising:
a first strand comprising, in the 5′ to 3′ direction, SEQ ID NO: 5, optionally an index, SEQ ID NO: 1, and 3SpC3; and
a second strand comprising, in the 5′ to 3′ direction, a 5′ block, SEQ ID NO: 2, optionally an index, and SEQ ID NO: 6. SEQ ID NOs: 1, 2, 5, and 6 may each comprise from 1 to 7, 1 to 6, 1 to 5, 1 to 4, 1 to 3, 2, or 1 modifications such as substitutions, deletions, or insertions. In an embodiment, the modifications are substitutions.

In a particular embodiment, the non-hairpin adapter is a Y-adapter comprising:
a first strand comprising, in the 5′ to 3′ direction, SEQ ID NO: 7, optionally an index, SEQ ID NO: 3, and 3SpC3; and
a second strand comprising, in the 5′ to 3′ direction, a 5′ block, SEQ ID NO: 4, optionally an index, and SEQ ID NO: 8. SEQ ID NOs: 3, 4, 7 and 8 may each comprise from 1 to 7, 1 to 6, 1 to 5, 1 to 4, 1 to 3, 2, or 1 modifications such as substitutions, deletions, or insertions. In an embodiment, the modifications are substitutions.

The 5′ and 3′ protective features may be any as disclosed for the first aspect of the present disclosure.

In some embodiments a non-hairpin adapter is or comprises nucleic acid. In some embodiments, the non-hairpin adapter is or comprises DNA, RNA, and/or XNA. The non-hairpin adapter may comprise modified and/or un-modified nucleotides. In some embodiments, the non-hairpin adapter is double-stranded. In a particular embodiment, the non-hairpin adapter comprises double-stranded DNA. The non-hairpin adapter may be a Y-adapter.

The non-hairpin adapter may comprise any 5′ and/or any 3′ binding feature as disclosed in relation to the first aspect of the present disclosure. The non-hairpin adapter may comprise or may not comprise any index as disclosed for the first aspect of the present disclosure.

In a particular embodiment, the non-hairpin adapter is a Y-adapter that comprises: a first strand comprising, in the 5′ to 3′ direction, a first hybridisation site to which a first sequencing primer can bind, a sequence that is at least partially complementary to a first immobilised primer, and a 3′ protective feature; and a second strand comprising, in the 5′ to 3′ direction, a 5′ protective feature, a sequence that is identical to at least a region of a second immobilised primer, and a second hybridisation site to which a second sequencing primer can bind. Optionally the first and second hybridisation sites are at least partially complementary.

In an eight aspect of the invention, there is provided a kit comprising a non-hairpin adapter of the seventh aspect of the present disclosure and a hairpin adapter. The hairpin adapter may be any as disclosed for the first aspect of the present disclosure.

TABLE 1

| Oligo modification (P5 or P7 adaptor) | Activity |
|---|---|
| Phosphorothioate linkages (5′ and 3′ | Resist 5′ > 3′ and 3′ > 5′ exonuclease activity. |
| ends of oligo) | |
| Phosphorothioate linkages (throughout | Resist endonuclease activity. |
| oligo) | |
| C3 Spacer phosphoramidite (3′) | Resist exonuclease, ligase, terminal transferase, |
| | 5′ > 3′ polymerase activity. |
| 3′ Phosphate group | Inhibit degradation by some 3′-exonucleases, can |
| | be used to block extension by DNA polymerases, |
| | and resist ligation. |
| 2′-O-Methyl (2′OMe) | DNA oligonucleotides that include this modification |
| | are typically 5- to 10-fold less susceptible to DNases |
| | than unmodified DNA. |
| Inverted dT (3′) | Inverted dT at 3′ end of an oligonucleotide leads to |
| | a 3′-3′ linkage that will inhibit degradation by 3′ |
| | exonucleases and extension by DNA polymerases. |
| 5′ Inverted ddT | 5′ Inverted ddT at the 5′ end of an oligonucleotide |
| | resists ligation and may provide resistance to some |
| | forms of exonuclease. |
| Locked nucleic acid bases | Increases melting temperature of oligo and |
| | prevents spurious hybridisation, increases binding |
| | specificity. |
| Dideoxycytidine (ddC) (3′) | 3′ chain terminator that prevents 3′ extension by |
| | polymerases. |

DEDUCE-seq adapters: Version 1-P7 hybridizes to flow cell at 3′ end
Bold underlined sequence: Complementary region of Y adapter

| | Y-adapter | | DNA insert | | Hairpin adapter |
|---|---|---|---|---|---|
| | | | | | DDD |
| 5′ | ′Block/A*ATGATACGGCGACCACCGAGATCTACAC[i5]ACACTCTTTCCCTACACGACGCTCTTCCGATC*T | 3′ | NNNNNNA | 5′ | P-GGGCCTA D |
| 3′ | /3SpC3/GTTCGTCTTCTGCCGTATGCTCTA[i7]CACTGACCTCAAGTCTGCACACGAGAAGGCTAG-P | 5′ | ANNNNNN | 3′ | T*CCCGGAT D |
| | | | | | DDD |

DEDUCE-seq adapters: Version 2-P5 hybridizes to flow cell at 3′ end
Bold underlined sequence: Complementary region of Y adapter

| | Y-adapter | | DNA insert | | Hairpin adapter |
|---|---|---|---|---|---|
| | | | | | DDD |
| 5′ | /5′Block/C*AAGCAGAAGACGGCATACGAGAT[i7]GTGACTGGAGTTCAGACGTGTGCTCTTCCGATC*T | 3′ | NNNNNNA | 5′ | P-GGGCCTA D |
| 3′ | /3SpC3/TTACTATGCCGCTGGTGGCTCTAGATGTG[i5]TGTGAGAAAGGGATGTGCTGCGAGAAGGCTAG-P | 5′ | ANNNNNN | 3′ | T*CCCGGAT D |
| | | | | | DDD |

Linear structure-once denatured and loaded on to Illumina flow cell
DEDUCE-seq adapters: Version 1-P7 hybridizes to flow cell at 3′ end

| | 5′ P5 adapter | | DNA insert | | Linear Hairpin adapter | |
|---|---|---|---|---|---|---|
| 5′ | ′Block/A*ATGATACGGCGACCACCGAGATCTACAC[i5]ACACTCTTTCCCTACACGACGCTCTTCCGATC*T | 3′ | NNNNNNA | 5 ′ | P-GGGCCTADDDDDDDDTAGGCCC*T | 3′ |

DEDUCE-seq adapters: Version 2-P5 hybridizes to flow cell at 3′ end

| | 5′ P7 adapter | | DNA insert | | Linear Hairpin adapter | |
|---|---|---|---|---|---|---|
| 5′ | /5′Block/C*AAGCAGAAGACGGCATACGAGAT[i7]GTGACTGGAGTTCAGACGTGTGCTCTTCCGATC*T | 3′ | NNNNNNA | 5′ | P-GGGCCTADDDDDDDDTAGGCCC*T | 3′ |

| | DNA insert | | 3′ P7 adapter |
|---|---|---|---|
| 3′ | NNNNNNA | 5′ | P-GATCGGAAGAGCACACGTCTGAACTCCAGTCAC[i7]ATCTCGTATGCCGTCTTCTGCTTG/3SpC3/ |
| | DNA insert | | 3′ P5 adapter |
| 3′ | NNNNNNA | 5′ | P-GATCGGAAGAGCGTCGTGTAGGGAAAGAGTGT[i5]GTGTAGATCTCGGTGGTCGCCGTATCATT/3SpC3/ |

DEDUCE-seq adapters (all 5′-3′)
Version 1-P7 hybridizes to flow cell at 3′ end

| | | |
|---|---|---|
| 5′ | /5′Block/A*ATGATACGGCGACCACCGAGATCTACA [i5]ACACTCTTTCCCTACACGACGCTCTTCCGATC*T | 3′ |
| 5′ | P-GATCGGAAGAGCACACGTCTGAACTCCAGTCAC[i7]ATCTCGTATGCCGTCTTCTGCTTG/3SpC3/ | 3′ |

Version 2-P5 hybridizes to flow cell at 3′ end

| | | |
|---|---|---|
| 5′ | /5′Block/C*AAGCAGAAGACGGCATACGAGAT[i7]GTGACTGGAGTTCAGACGTGTGCTCTTCCGATC*T | 3′ |
| 5′ | P-GATCGGAAGAGCGTCGTGTAGGGAAAGAGTGT[i5]GTGTAGATCTCGGTGGTCGCCGTATCATT/ 3SpC3/ | 3′ |

Hairpin

| | | |
|---|---|---|
| 5′ | P-GGGCCTADDDDDDDDTAGGCCC*T | 3′ |

| Stem | | Full Sequence + potential derivatives |
|---|---|---|
| 7 | bp | 5′ P-GGGCCTADDDDDDDDTAGGCCC*T 3′ |
| Loop | | STEM Loop STEM T-tail |
| 8 | bp | |
| | | DDD |
| | | 5′ GGGCCTA D |
| | | 3′ T*CCCGGAT D |
| | | DDD |
| | | D = G, A or T (not C) = |

*Phosphorothioate linkage Resistance to exonuclease activity
/3SpC3/C3 Spacer phosphoramidite (covalent block)
Resistance to exonuclease, ligase, and 5′ > 3′ polymerase activity
-P 5′ Phosphate group Facilitate 5′ ligation
INDEX Illumina sequencing index sequence Enable demultiplexing of pooled sequencing libraries
*T 3′ deoxythymidine triphosphate ′T-tail′ with phosphorothioate linkage Provide substrate for ligation to ′A-tailed′ DNA fragments

The sequences on pages 44 to 47 are:
Y-adapter version 1 (in order): SEQ ID NOs: 4, 8, 9, 7, and 3.
Y-adapter version 2 (in order): SEQ ID NOs: 2, 6, 9, 5, and 1.
Hairpin: SEQ ID NO: 9

### EXAMPLES

### Example 1—DEDUCE-Seq

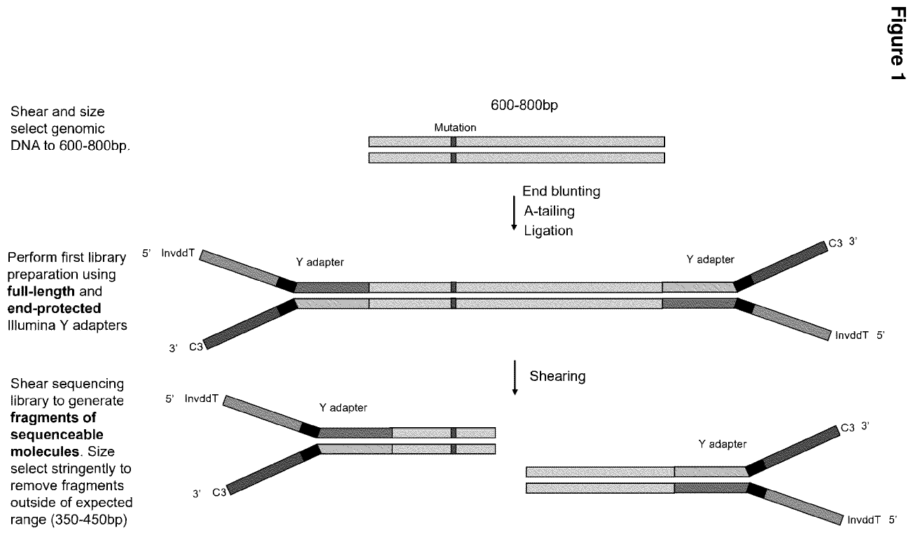

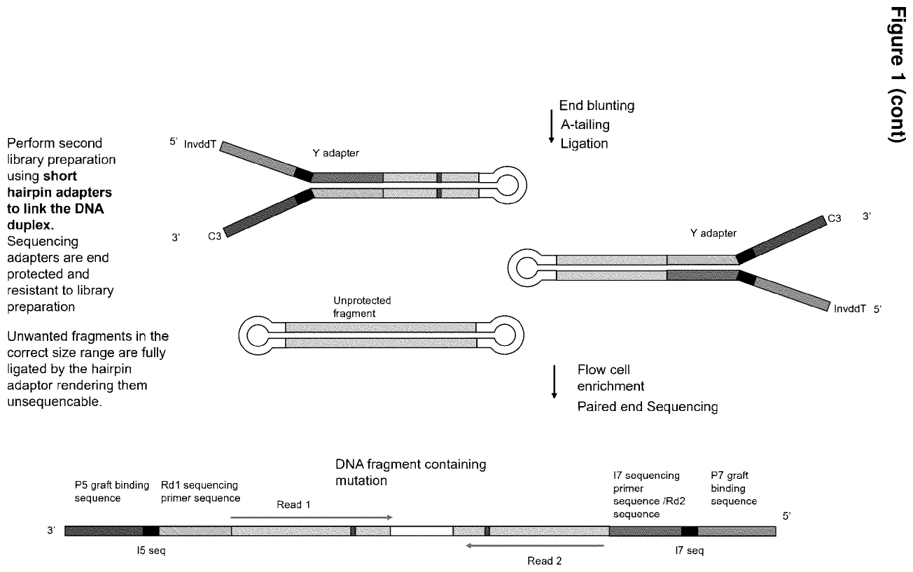

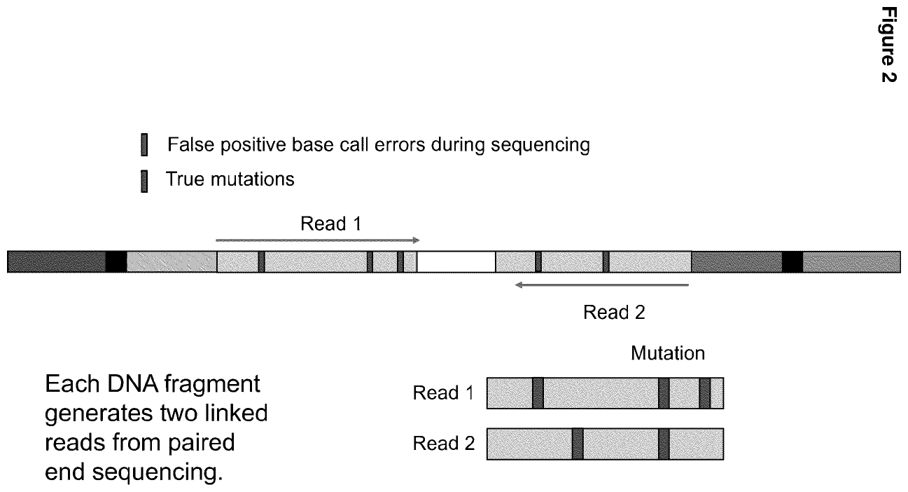

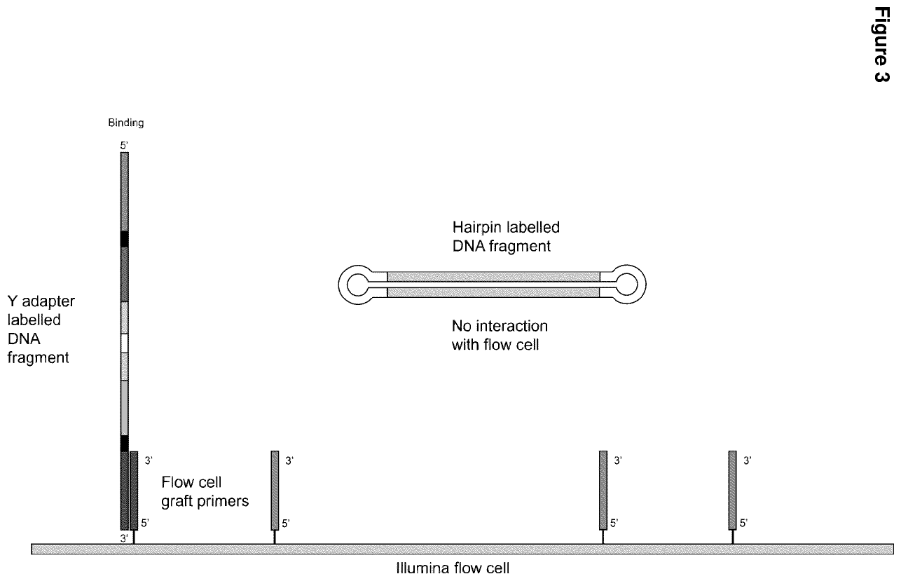

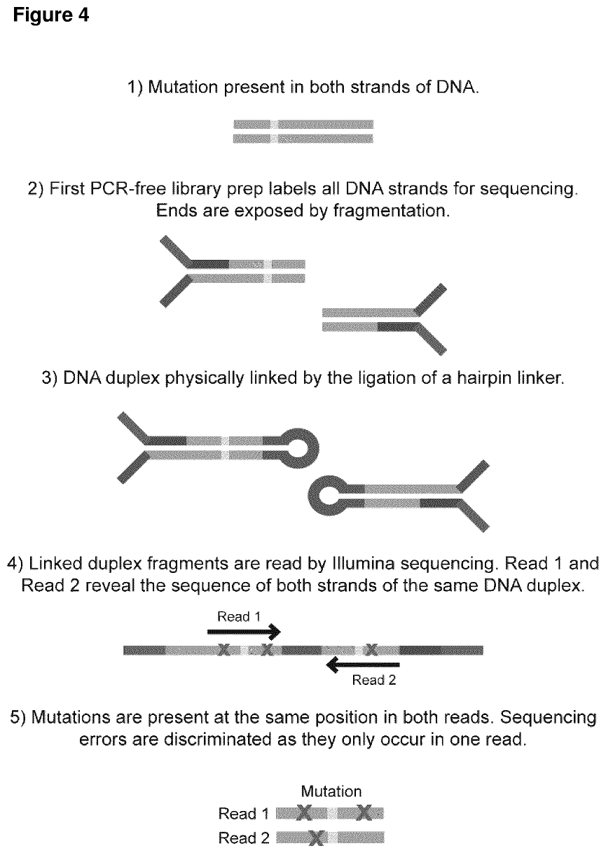

DuplEx Determination by Unbiased flow Cell Enrichment and sequencing (DEDUCE-seq) uses a full-length Y-adapter to build in all the necessary DNA elements required for Illumina sequencing, while a second hairpin adapter will lock the oligo and link both strands thus retaining duplex information a linear molecule (FIG. 1). As with the Duplex-seq method [Kennedy, S. R., et al., Detecting ultralow-frequency mutations by Duplex Sequencing. Nat Protoc, 2014. 9(11): p. 2586-606], which retains duplex information using a tag-based system, DEDUCE-seq exploits the complementary nature of DNA to discriminate between genuine mutations and sequencing errors. However, DEDUCE-seq achieves this by physically linking both strands of the DNA duplex into a single sequencable DNA molecule. Moreover, general base-calling accuracy of current sequencers has increased by at least an order of magnitude in the last decade (to 1 in 10), improving the theoretical limit at which variants can be called. By using the redundant information from both DNA strands, only true mutations or variants will be called on both strands of the duplex, whereas technical errors can be eliminated as they will exist in only one of the two reads (FIG. 1). This strategy has been shown to greatly enhance the accurate detection of ultra-rare mutations (down to 1×10⁻⁶) [Salk, J. J. and S. R. Kennedy, Next-Generation Genotoxicology: Using Modern Sequencing Technologies to Assess Somatic Mutagenesis and Cancer Risk. Environ Mol Mutagen, 2020. 61(1): p. 135-151]. However, Duplex-seq relies on Unique Molecular Identifiers (UMI's) and PCR amplification to achieve this [Kennedy, S. R., et al., Detecting ultralow-frequency mutations by Duplex Sequencing. Nat Protoc, 2014. 9(11): p. 2586-606]. This method is therefore not PCR-free, greatly increasing the cost of sequencing, and limiting its application to targeted sequencing. DEDUCE-seq, however, is PCR-free by design and instead uses the Illumina flow cell for DNA enrichment and is designed to be able to cost-effectively detect rare mutations genome-wide.

In the first instance, the inventors will use DEDUCE-seq to detect mutations from an isogenic yeast experiment previously conducted. In this project, a mutational survey was performed of multiple yeast strains that were treated with UV irradiation, after which cells were propagated for ~1,200 generations to accumulate mutations. The mutations acquired during these experiments were measured using traditional WGS and variant calling. This legacy data of the small-sized, yeast genome, therefore, allows DEDUCE-seq to be benchmarked for in vivo mutation detection. Next, DEDUCE-seq will be applied for the detection of mutations at novel off-target sites discovered by INDUCE-seq (WO2022/038291 A1). CRISPR genome editing projects are available for the detection of mutations in human cells. Using INDUCE-seq the inventors have discovered novel off-targets for very strict guide RNAs and very poorly targeting ones, allowing DEDUCE-seq to assay both ends of this spectrum to evaluate mutation induction at high- and low-frequency off-target break sites in human cells.

### Methods—Initial Design

The inventors will make use of genomic DNA from a mutation survey performed in yeast (see above). To establish a library preparation, genomic DNA will be fragmented to a size of ~600-800 bp. The first ligation uses a full-length Y-adapter to build in all the adapter components required for sequencing (FIG. 1). Next, the DNA is purified to remove excess adapter DNA and subjected to a second round of fragmentation to ~200-300 bp. DNA size selection, successful ligation and removal of adapter DNA will all be assessed using capillary DNA electrophoresis. For the second ligation the inventors will use a hairpin adapter to physically link the complementary strands of DNA and lock the duplex information into a single sequencable molecule (see FIG. 1). Size-selection and purification of this library DNA will also remove excess hairpin adapter DNA. To validate the successful ligations, qPCR will be used to quantify the sequencable DNA fraction of the sample. This will reveal that the design of the adapters shown in FIG. 1 results in functional, sequencable molecules. These pilot experiments will first be paired-end sequenced on a small scale. This will confirm the successful ligation strategy and reveal the general quality of the library. For this the inventors will initially sequence DNA from untreated cells (low mutation rate) and highly mutagenic cells (high mutation rate). The preliminary data from these experiments will be used to start the development of a data analysis pipeline that exploits duplex information. Next, the pilot DEDUCE-seq experiments will be scaled up to more samples and higher coverage (~100×) using a high-capacity sequencing platform (MiSeq v3 or NextSeq 550) to detect mutations in early- and late-generation yeast from (i) untreated wildtype cells, (ii) UV irradiated wildtype cells and (iii) cells with a known mutator phenotype. Finally, after these pilot experiments the inventors will apply DEDUCE-seq for the detection of mutations from a large cohort of yeast samples of the above-described mutagenesis project previously conducted. Data generated from this can now be used to assess the performance of DEDUCE-seq compared to original WGS performed at ~10-25× coverage. Once established, the method will be used to detect genome-wide mutations from CRISPR-Cas9 edited genomic DNA with low- and high frequency off-targets as measured by INDUCE-seq.

### Methods—for Pilot 1 and Pilot 2

### Genomic DNA Input

To generate DEDUCE-seq libraries, fragmented genomic yeast DNA was used as input. The genomic DNA samples were defrosted and run on an automated electrophoresis system (Agilent TapeStation 2100, High Sensitivity D1000 screentape) to assess size-distribution and quality. Next, the DNA was quantified using a Qubit-2 (ThermoFisher) using the high sensitivity kit (Qubit™ dsDNA HS Assay Kit) and normalised to 200-250 ng per 50 μL in nuclease free water (NFW).

### DEDUCE-Seq Library Preparation

Genomic DNA was prepared using a 1-sided size-selection. First, 0.6× (v/v) SPRI beads (CleanNGS, GCBiotech) removes fragments larger than 300 bp, maintaining the DNA of interest from 100 to 500 bp in solution. In the second purification step, SPRI beads were added to a final concentration of 1.8× (v/v) and DNA was eluted to a final volume of 25 μL NFW. Next, the DNA was blunt ended and A-tailed using the NEBNext® Ultra™ II End Repair/dA-Tailing Module (E7546L, New England Biolabs) in an end volume of 30 μL, ready for ligation using the NEBNext® Ultra™ II Ligation Module (E7595L, New England Biolabs). For the first ligation, Pilot-1 used 1.25 μL 7.5 μM full length Y-adapter (P5-P7), while Pilot-2 used 1.25 μL 7.5 μM of hairpin adapter. Total DNA was purified, and remaining adapter removed using 1.8× (v/v) SPRI beads, after which the DNA was eluted in 100 μL NFW ready for sonication. The ligated DNA was subjected to resonication using a Bioruptor (Diagenode) for 60 cycles (30 seconds on/off, high output). To prepare for the second round of end-prep and ligation, the DNA was purified using 1.8× (v/v) SPRI beads and eluted in 25 μL NFW to reduce the volume suitable for the NEBNext Ultra II modules. The resonicated DNA was blunt-ended and A-tailed using the NEBNext® Ultra™ II End Repair/dA-Tailing Module (E7546L, New England Biolabs) and ligated using the NEBNext® Ultra™ II Ligation Module (E7595L, New England Biolabs), as described above. In Pilot-1 1.25 μL 7.5 μM hairpin adapter was used, in Pilot-2, 1.25 μL 7.5 μM of full-length Y-adapter (P5-P7) was used in the second ligation. After the second and final ligation, DNA was purified using 1.8× (v/v) SPRI beads and eluted in 28 μL NFW. The final libraries were quantified using qPCR and tested on an automated electrophoresis system (Agilent TapeStation 2100, high sensitivity D1000 screentape) to assess size-distribution and quality. Throughout the protocol, the size and quality of the library DNA was measured using electrophoresis to assess adapter removal, resonication and final library.

### DEDUCE-Seq Library Quantification

Final DEDUCE-seq library DNA was diluted 50-fold in dilution buffer (10 mM Tris-HCl, pH 8.0, 0.05% Tween-20) and 4 μL of diluted library DNA was subjected to qPCR in triplicate using a final PCR reaction volume of 20 μL (KAPA Library Quantification Kit Illumina® Platforms). The library DNA was amplified using the cycling protocol as recommended by the supplier's guidelines and quantified using the supplied tools to obtain the undiluted library concentration (μM).

### Sequencing of DEDUCE-Seq Libraries

Final DNA libraries were pooled where relevant and the final volume reduced to 40 μL using a SpeedVac. Before loading onto the sequencing flow cell, the DEDUCE-seq libraries are prepared according to the following modified denaturing protocol: the final library (40 L) is combined with 40 μL of freshly diluted 0.2 N NaOH at room temperature for 5 minutes to denature the DNA. Next, 40 μL of 200 mM Tris-HCl (pH 7) is used to neutralise the solution. The resulting denatured library (120 μL) is complemented with 1179 L of prechilled HT1 and 1 μL of denatured and diluted PhiX control (20 pM). This mixture of 1.3 mL is loaded onto the NextSeq cartridge for sequencing in its entirety.

### Sequencing Data Processing

Sequencing runs were assessed using the Illumina's online basespace utility or offline Sequence Analysis Viewer (SAV, Illumina). Reads pass filter, base-call quality (Q30) and cluster density are used as a first pass quality control. Demultiplexed data is then retrieved, ready for downstream analysis, described blow.

### Secondary Data Analysis

Demultiplexed sequencing data was downloaded from basespace as FASTQ files. Using trim_galore (v0.6.7) reads were quality and adapter trimmed with standard parameters. FASTQC was used to quality check the trimmed and untrimmed data. To retrieve HP containing reads standard command-line tools GNU grep (3.7) and AWK (1.3.4) were used to interrogate the data. Seqkit fq2fa was first used to convert the FASTQ files to FASTA format, before locate was applied for calculating the exact position of hairpin sequence in Reads 1 and 2 using the following commands:

seqkit fq2fa -j $threads $Read1 -o $Read1.fa.gz
seqkit locate -j $threads -i -d -P -p AGGGCCTANNNNNNNNTAGGCCC $Read1. fa.gz
>$Reads1_HP-locate.tsv

Alignment of DEDUCE-seq data was performed using bowtie2 (2.5.1) using default parameters for exploratory analysis aligning concordant read pairs and for discordant DEDUCE-seq reads in the following ways:

# default concordant alignment
bowtie2 −p $threads −x $refseq −1 $mate1 −2 $mate2
# DEDUCE-seq discordant alignment
bowtie2 −p $threads −−ff −−no-mix −−dovetail −x $refseq −1 $mate1 −2
$mate2

Where relevant unmapped reads and secondary alignments were removed using samtools (1.6):
samtools view -Shu -f 3 -F 256 -@$threads $input

Aligned data was converted to BAM files using samtools (1.6) and visualised using the Intergrated Genomics Viewer (2.14.1) (Robinson et al. 2011).

### Example 2—DEDUCE-Seq Pilot 1 and Pilot 2

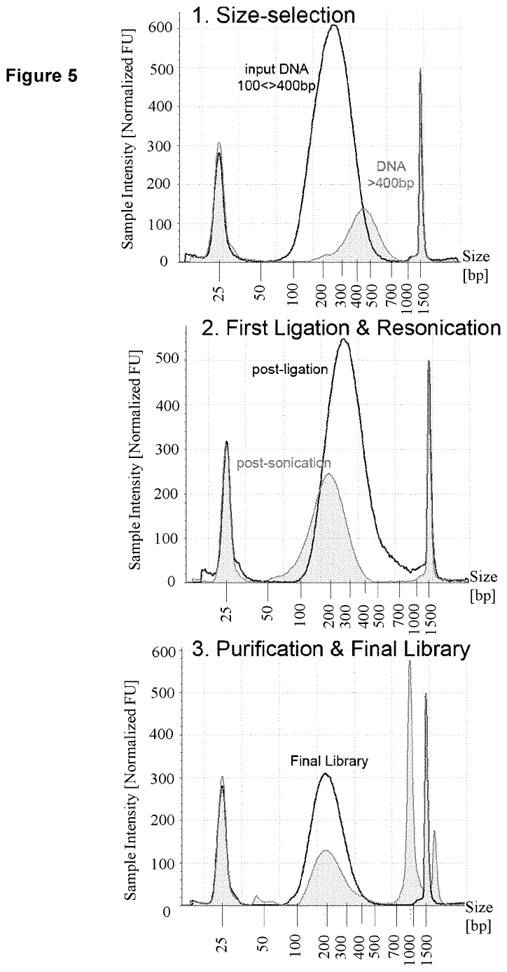

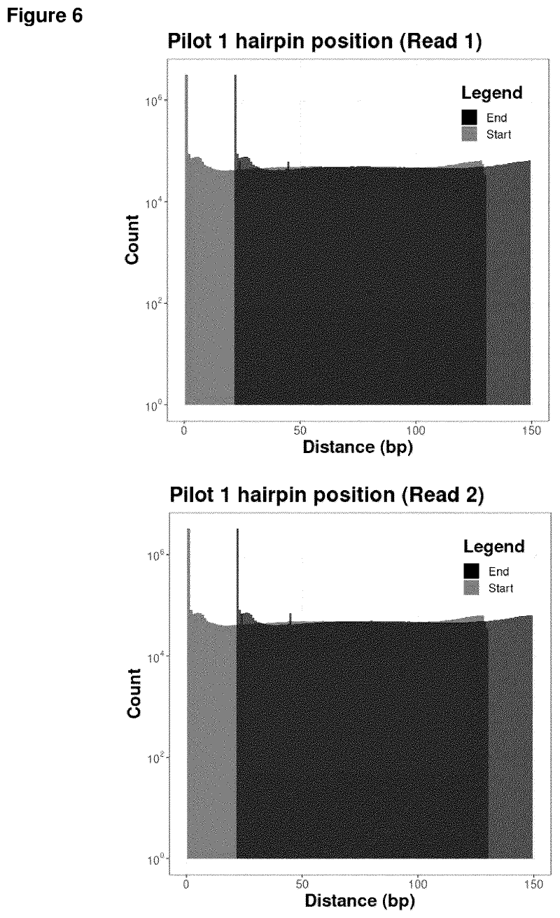

The pilot studies described here were designed to establish the core elements of the DEDUCE-seq library and determine the most efficient ligation strategy. Therefore, we generated DEDUCE-seq libraries with the hairpin ligated first and the Y-adapter second (Pilot-1) and vice versa (Pilot-2). In these studies, genomic yeast DNA was used to generate DEDUCE-seq libraries. This DNA was previously used to measure mutations in a study designed to detect UV irradiation-induced mutations in isogenic yeast strains (Nandi et al. 2018) and provides a suitable source of genomic DNA of known origin with a known mutation burden. These samples are stored as fragmented DNA of ~200-300 bp and normalised to 4-5 ng/μL. First, for Pilot-1 2 samples were processed in parallel and subjected to a right-sided size-selection to remove larger DNA fragments>300 bp (data for one representative sample shown). The starting DNA ranges from 100 to 500 bp (FIG. 5, left panel).

### Pilot 1—DEDUCE-Seq Hairpin Ligated First and the Y-Adapter Second—Library Construction

Next, the DNA was blunted, A-tailed, and the hairpin adapter was ligated using the NEBNext Ultra II kits. The ligated DNA was purified and checked on TapeStation to confirm removal of the hairpin adapter DNA (FIG. 5, middle panel, black trace). Resonicating the DNA results in a shift of the size distribution centred on ~200 bp ranging from 75 to 500 bp (FIG. 5, middle panel, grey trace). The end-prep and ligation process were repeated for the second Y-adapter, resulting in a final purified library shown in FIG. 5 (right panel, grey trace). The final ligation does not result in a major shift of the size distribution. Residual Y-adapter can be detected at 50 bp, and high molecular weight fragments are detected after Y-adapter ligation around 900 bp (FIG. 5, right panel). Importantly, this is a known artifact of Y-adapter ligation shown previously as part of the Duplex-seq methodology manuscript using a stubby Y-adapter (Kennedy et al. 2014). This large fraction can be safely ignored.

The final library contains a mixture of molecules of which one fraction is made up of functional DEDUCE-seq HP-Y-adapter ligated fragments. Due to the presence of the full-length P5-P7 hybrid Y-adapter, DEDUCE-seq library molecules contain the constituent primer binding sites required for quantification. We therefore applied qPCR to quantify the sequencable molecules in the final library prep and measured 1.1 and 2.0 nM library concentrations for samples 1 and 2, respectively. Importantly, the high molecular weight artifacts shown in FIG. 5 (right panel) do not contribute to the qPCR readout. No molecules with an exceedingly high melting temperature can be detected in these samples (data not shown). The final libraries are predicted to contain between 190 and 355 million sequencable molecules per 1 μL of undiluted library for sample 1 and 2, respectively, at these concentrations. Therefore, 1 μL of the library from sample 2 was sequenced on a NextSeq 500 High output 2×150 bp flow cell resulting in 240 million reads of which 96% passed filter with a Q30 score of 93%. Successful sequencing demonstrated that the HP-Y-adapter ligation strategy of Pilot-1 resulted in functional, sequencable molecules that can be quantified by qPCR and loaded onto a sequencer to generate clusters and reads proportional to the qPCR readout.

### Pilot 1—DEDUCE-Seq Hairpin Ligated First and the Y-Adapter Second—Data Analysis

The design of the DEDUCE-seq library is non-standard and is predicted to result in discordant read pairs in the Forward-Forward (F1F2) or Reverse-Reverse (R2R1) orientation that not all aligners accept as legitimate output. Similarly, dovetailed reads can result from this library depending on insert length and trimming and are not accepted by all aligners. Furthermore, we found from preliminary alignments experiments that most reads aligned as concordant pairs (F1R2 or R1F2) and surmised that these were derived from double Y-adapter ligated product, which was predicted as a minority DEDUCE-seq output (data not shown). Therefore, we first assessed the composition of the DEDUCE-seq sequencing reads by searching for hairpin containing reads in an unbiased way. Programmatically retrieving hairpin containing reads from read 1 and read 2 returned 33 and 44M reads (14-18.4%) from a total of 237M reads, respectively (Table 2). Not every duplex molecule is expected to contain HP sequence in the reads if the genomic DNA insert size is larger than 150 bp. Interestingly, basic positional information from this search revealed that most reads contain HP sequence at the start of the read, whereas around 7.5M reads contain HP sequence somewhere in the middle of the read. However, rarely do we find HP sequence at the extreme end of a read (Table 2).

TABLE 2

| | Read 1 | Read 2 |
|---|---|---|
| Reads with no Hairpin | 204,467,929 | 193,705,101 |
| Reads with Hairpin | 33,297,970 | 44,060,798 |
| @Read Start | 25,611,951 | 36,501,713 |
| @Read Middle | 7,648,190 | 7,523,125 |
| @Read End | 37,829 | 35,960 |
| Total | 237,765,899 | 237,765,899 |

With this information we extracted all read pairs that contain the HP sequence in both R1 and R2 to elucidate the conformation of these molecules. Using this list of 9.5M read pairs we calculated the exact position of the hairpin sequence in each read and plotted the distribution of hairpin position as a function of read length (<151 bp).

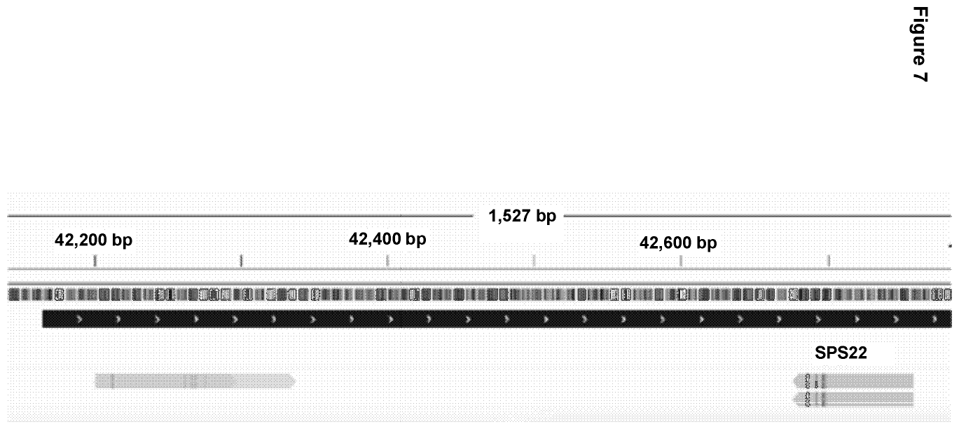

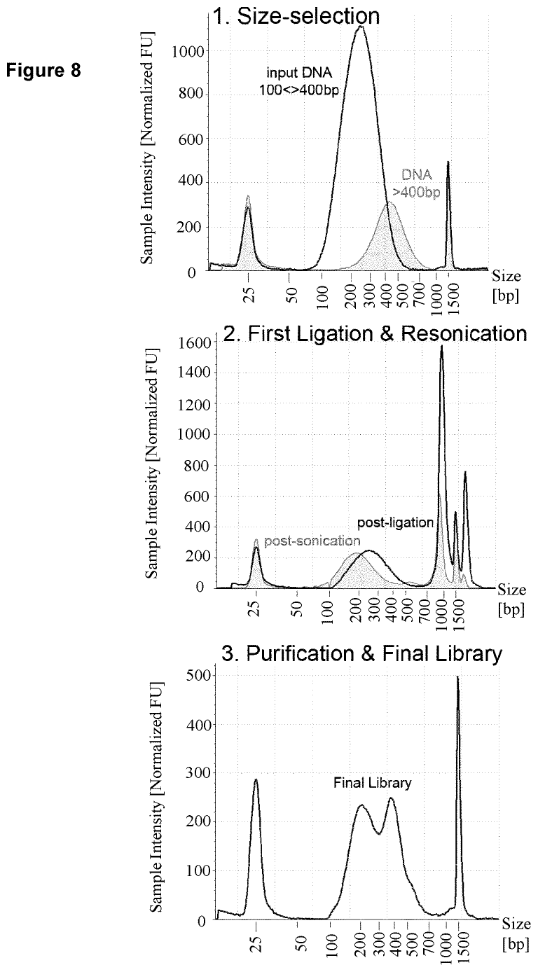

We find that for this class of reads, about half contain hairpin DNA towards the start of the read, as shown in Table 2. In the remaining reads, the HP sequence is distributed evenly across the read length. Next, we collected the 9.5M read pairs (4%) containing HP sequence and aligned them to the reference genome using bowtie2. Bowtie2 can accommodate discordant read pairs enabling us to align about 200-800 reads and inspect them in a genome browser (FIG. 7). This revealed the correct double parallel orientation (F1F2 and R1R2) for all of these reads as expected from the DEDUCE-seq library design. This confirmed that HP containing reads can be aligned using bowtie2 and are in the correct orientation, further revealing the associated SAM flags that define these read pairs (F1F2, SAM flag 67-133 and R2R1, SAM flag 115-179).

With this information we aligned the entire dataset of 230M reads using the specific configuration of bowtie2 described above, and used the corresponding SAM flags to filter out the correctly aligned DEDUCE-seq reads. This resulted in ~120K reads with the expected DEDUCE-seq-specific orientation (FIG. 7 and Table 3), indicating that the combination of a hairpin and Y-adapter ligation results in a functional library. The results of this alignment are summarised in Table 4.

TABLE 3
DEDUCE-seq Library Discordant Read Pairs

| | Left | Right | Left | Right |
|---|---|---|---|---|
| | alignment | alignment | alignment | alignment |
| Flags | 67 | 131 | 115 | 179 |
| Mapping Quality | 40 | 40 | 42 | 42 |
| CIGAR | 92M | 133M | 79M | 79M |
| Mate is Mapped | yes | yes | yes | yes |
| Position | First in | Second in | First in | Second in |
| | Pair | Pair | Pair | Pair |
| Pair Orientation | F1F2 | F1F2 | R2R1 | R1R2 |

TABLE 4

| Reads | | | Flag | Description |
|---|---|---|---|---|
| Read 1 | 197,911,359 | 197,8520,805 | 77 | paired \| unmapped \| mate unmapped \| |
| | | | | 1st |
| Read 2 | 197,911,359 | 197,8520,805 | 141 | paired \| unmapped \| mate unmapped \| |
| | | | | 2nd |
| properly paired with | 181,108 | 38,869 | 67 | paired \| fwd \| mapped in proper pair \| |
| itself and mate | | | | 1st |
| mapped | | 38,869 | 131 | paired \| fwd \| mapped in proper pair \| |
| | | | | 2nd |
| | | 43,857 | 115 | paired \| rev \| mapped in proper pair \| |
| | | | | 1st |
| | | 43,857 | 179 | paired \| rev \| mapped in proper pair \| |
| | | | | 2nd |
| | | 3,555 | 65 | paired \| fwd \| 1st |
| | | 3,555 | 129 | paired \| fwd \| 2nd |
| | | 4,273 | 113 | paired \| rev \| 1st |
| | | 4,273 | 177 | paired \| rev \| 2nd |

Forcing the alignment of DEDUCE-seq data through bowtie2 in this way results in >190M read pairs as unmapped, which would normally (based on the SAM flags) result in concordant pairs (data not shown). Conversely, the properly paired and mapped reads fall into the correct classes of discordant, parallel reads (F1F2 & R2R1) adding up to 180K (Table 2).

### Pilot-2 DEDUCE-Seq Ligating Y-Adapter First, Hairpin Second: Library Construction

Similar to Pilot-1, the DEDUCE-seq library for Pilot-2 was derived from the same genomic DNA. In this instance a total of 250 ng of DNA from 4 independent samples was size selected to remove large fragments of DNA (>500 bp) and prepared for ligation. In the first round the full-length Y-adapter was ligated onto the DNA. After purification and removal of unligated Y-adapter, the DNA was resonicated for 60 cycles and purified. The DNA was processed through another round of end-prep and ligation to attach the hairpin adapter after which the DNA was purified and quantified using qPCR. The final library concentration of these samples ranged between 2.8 to 8.3 pM.

Thus, preparing a DEDUCE-seq library by ligating the Y-adapter first and hairpin adapter second, results in a yield of sequencable molecules that is about 3 orders of magnitude lower than the reverse order performed in Pilot-1. This demonstrates that the efficiency of ligation between Y- or hairpin adapter is distinct, and that the order of ligation affects the yield of the DEDUCE-seq library. The estimated sequencing reads from the samples prepared in Pilot-2 range from 11 to 35M. Therefore, we pooled the 4 samples together for a total of 73M predicted reads and sequenced the pool on a NextSeq 500 High output 2×150 bp flow cell. This sequencing run resulted in 38 million reads of which 91% passed filter with a Q30 score of 91%. Importantly, the DEDUCE-seq library generated here, resulted in a lower sequencing output than expected from the qPCR quantification. The original design of a DEDUCE-seq library constructed in this way, facilitates the flow cell enrichment of correctly ligated Y-DNA-Hairpin products, while simultaneously locking unligated fragments into a double hairpin-ligated circle rendering these molecular inert. This would theoretically improve the selective enrichment of sequencable molecules on the flow cell.

### Pilot-2 DEDUCE-Seq Ligating Y-Adapter First, Hairpin Second: Data Analysis

Based on the findings derived from Pilot-1, we used the same approach described above and collated the hairpin containing reads to calculate the position of hairpin sequence in each read.

TABLE 5

| | Read 1 | Read 2 |
|---|---|---|
| Reads with no Hairpin | 38,737,660 | 38,750,812 |
| Reads with Hairpin | 474,893 | 422,286 |
| @Read Start | 64 | 70 |
| @Read Middle | 466,201 | 414,949 |
| @Read End | 8,628 | 7,267 |
| Total | 38,856,384 | 38,856,384 |

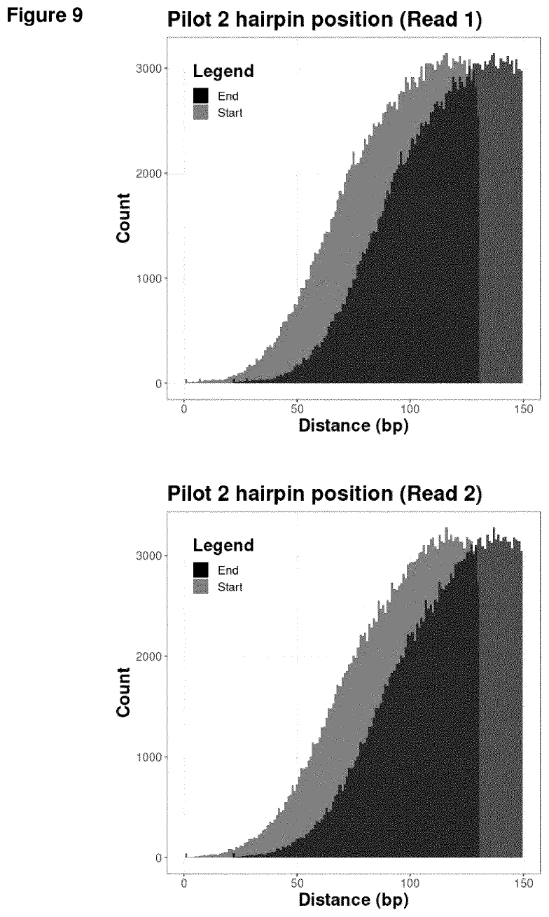

Taken together this returned between 420-470K reads from both R1 and R2 containing hairpin sequence from a total of 38M read pairs (1.1-1.2%). Combining pairs where both reads contain HP sequence leaves 200K read pairs (~0.5%) of which 23K align to the reference genome (~0.05%). The distribution of hairpin positions is shown in FIG. 9. Ligating hairpin adapter second results in the majority of HP sequence to be positioned towards the 3′ or end of read 1 or read 2 as expected.

Importantly, this alignment was performed in the presence of non-coding hairpin sequence within Reads 1 and 2 that does not exist in the yeast reference genome interfering with the aligner. Trimming the hairpin DNA from these reads improves the alignment (data not shown).

### CONCLUSION

Taken together both orientations of Y- and HP-adapter ligation of DEDUCE-seq result in parallel, duplex molecules as per the DEDUCE-seq design.

Ligating the Y-adapter first and hairpin second, as done in Pilot-2, may be the preferred option to fully exploit flow cell enrichment of properly formed Y-HP molecules from double hairpin molecules that are inert. However, this library strategy is less efficient compared to that applied in Pilot-1. In Pilot-1 the total yield of the library is higher (nM) compared to Pilot-2 (pM).

1. A method of library preparation for nucleic acid sequencing, the method comprising:
a) providing a plurality of nucleic acids;
b) exposing the plurality of nucleic acids to a non-hairpin adapter under conditions conducive to ligation;
c) fragmenting the plurality of nucleic acids; and
d) exposing the plurality of nucleic acids to a hairpin adapter under conditions conducive to ligation, or exposing the plurality of nucleic acids to conditions capable of forming a hairpin at an end of a nucleic acid molecule;
wherein steps b) and d) are performed separately.

2. The method of claim 1, wherein the plurality of nucleic acids is fragmented after the first adapter ligation step and before, or as a part of, the second adapter ligation step.

3. The method of claim 1, wherein:
the steps are performed sequentially and in the order a), b), c), d); or
the steps are performed sequentially and in the order a), d), c), b); or
the steps are performed in the order step a), step b), and combined steps c) and d); or
the steps are performed in the order step a), step d), and combined steps c) and b).

4. The method of any one of claims 1 to 3, wherein the non-hairpin adapter comprises a sequence that is at least partially complementary to a first primer that is immobilised to a substrate.

5. The method of claim 4, wherein the sequence that is at least partially complementary to a first primer that is immobilised to a substrate comprises at least 5, 10, 15, 16, 17 18, 19, 20, or all 21 bases of SEQ ID NO: 1 or at least 5, 10, 15, 16, 17 18, 19, 20, 21, 22, 23, or all 24 bases of SEQ ID NO: 3.

6. The method of any one of claims 1 to 5, wherein the non-hairpin adapter is a Y-adapter.

7. The method of claim 6, wherein the Y-adapter comprises:
a first strand comprising a sequence that is at least partially complementary to a first primer immobilised to a substrate; and
a second strand comprising a sequence that is identical to at least a region of a second primer.

8. The method of claim 7, wherein the sequence that is identical to at least a region of a second primer comprises at least 5, 10, 15, 16, 17 18, 19, 20, 21, 22, 23, or all 24 bases of SEQ ID NO: 2 or at least 5, 10, 15, 16, 17 18, 19, or all 20 bases of SEQ ID NO: 4.

9. The method of any preceding claim, wherein the non-hairpin adapter is a Y-adapter that comprises:
a first strand comprising, in the 5′ to 3′ direction, a first hybridisation site to which a first sequencing primer can bind, and a sequence that is at least partially complementary to a first immobilised primer; and
a second strand comprising, in the 5′ to 3′ direction, a sequence that is identical to a region of a second immobilised primer and a second hybridisation site to which a second sequencing primer can bind.

10. The method of any preceding claim, wherein the non-hairpin adapter comprises a 5′ and/or a 3′ protective feature.

11. The method of claim 10, wherein the non-hairpin adapter comprises a first strand comprising a 3′ protective feature and a second strand comprising a 5′ protective feature.

12. The method of any preceding claim, wherein the non-hairpin adapter is a Y-adapter that comprises:
a first strand comprising, in the 5′ to 3′ direction, a first hybridisation site to which a first sequencing primer can bind, a sequence that is at least partially complementary to a first immobilised primer, and a 3′ protective feature; and
a second strand comprising, in the 5′ to 3′ direction, a 5′ protective feature, a sequence that is identical to at least a region of a second primer, and a second hybridisation site to which a second sequencing primer can bind.

13. The method of any preceding claim, wherein the plurality of nucleic acids is DNA.

14. The method of claim 13, wherein the plurality of nucleic acids is genomic DNA (gDNA).

15. The method of any preceding claim, wherein the method comprises:
e) contacting the plurality of nucleic acids to a substrate comprising a first immobilised primer under conditions suitable for hybridisation of the first immobilised primer to complementary nucleic acids;
wherein the non-hairpin adapter comprises a sequence that is at least partially complementary to the first immobilised primer.

16. The method of claim 15, wherein no nucleic acid amplification step is performed prior to step e).

17. The method of claim 15 or claim 16, wherein the non-hairpin adapter comprises a sequence that is identical to at least a region of a second primer and the second primer is immobilised to the substrate.

18. The method of claim 17, wherein the first and second immobilised primers are capable of acting as forward and reverse primers for bridge amplification, and wherein the method comprises bridge amplification.

19. The method of any one of claims 15 to 18, wherein the substrate is a flow cell or a bead.

20. The method of any one of claims 15 to 19, wherein the method comprises:
f) obtaining sequence information for any nucleic acids that hybridised to the substrate in step e).

21. The method of any one of claims 1 to 14, wherein, after steps a), b), c), and d) have been performed, the method further comprises obtaining sequence information from the prepared library.

22. A nucleic acid library obtained or obtainable by a method of any one of claims 1 to 14.

23. A method of sequencing, wherein the method comprises obtaining sequence information for nucleic acids within a library of claim 22.

24. A method of obtaining sequencing information, wherein the method comprises:
1) contacting a library of claim 22 to a substrate comprising a first immobilised primer under conditions suitable for hybridisation of the first immobilised primer to complementary nucleic acids; and
2) obtaining sequence information for any nucleic acids that hybridised to the substrate in step 1).

25. Use of a nucleic acid library of claim 22, or a nucleic acid library obtained or obtainable by a method of any one of claims 1 to 14, in a nucleic acid sequencing method.

26. A method of library preparation for nucleic acid sequencing, the method comprising:
i) providing a plurality of nucleic acids;
ii) exposing the plurality of nucleic acids to a non-hairpin adapter under conditions conducive to ligation; and
iii) exposing the plurality of nucleic acids to a hairpin adapter under conditions conducive to ligation, or exposing the plurality of nucleic acids to conditions capable of forming a hairpin at an end of a nucleic acid molecule;
wherein the nucleic acids are not amplified during preparation of the library.

27. The method of claim 26, wherein the method comprises:
iv) contacting the plurality of nucleic acids to a substrate comprising a first immobilised primer under conditions suitable for hybridisation of the first immobilised primer to complementary nucleic acids;
wherein the non-hairpin adapter comprises a sequence that is at least partially complementary to the first primer that is immobilised to the substrate; and
wherein the nucleic acids are not amplified prior to step iv).

28. The method of claim 27, wherein the non-hairpin adapter comprises a sequence that is identical to at least a region of a second primer and the second primer is immobilised to the substrate.

29. The method of claim 28, wherein the first and second immobilised primers are capable of acting as forward and reverse primers for bridge amplification, and wherein the method comprises bridge amplification.

30. The method of any one of claims 27 to 29, wherein the method comprises obtaining sequence information for any nucleic acids that hybridised to the substrate in step iv).

31. The method of any one of claims 26 to 30, wherein steps ii) and iii) are performed separately, and wherein a fragmentation step is performed after step ii) and before step iii) or after step iii) and before step ii).

32. A nucleic acid library obtained or obtainable by a method of claim 26.

33. A method of sequencing, wherein the method comprises obtaining sequence information for nucleic acids within a library of claim 32.

34. A method of obtaining sequencing information, wherein the method comprises:
1) contacting a library of claim 32 to a substrate comprising a first immobilised primer under conditions suitable for hybridisation of the first immobilised primer to complementary nucleic acids; and
2) obtaining sequence information for any nucleic acids that hybridised to the substrate in step 1).

35. Use of a nucleic acid library of claim 32, or a nucleic acid library obtained or obtainable by a method of claim 26, in a nucleic acid sequencing method.


SEQUENCE LISTING (9 sequences)

SEQ ID NO: 1 - DNA, 29 bp, synthetic construct
  source 1..29: mol_type = other DNA
gtgtagatctcggtggtcgccgtatcatt

SEQ ID NO: 2 - DNA, 24 bp, synthetic construct
  source 1..24: mol_type = other DNA
caagcagaagacggcatacgagat

SEQ ID NO: 3 - DNA, 24 bp, synthetic construct
  source 1..24: mol_type = other DNA
atctcgtatgccgtcttctgcttg

SEQ ID NO: 4 - DNA, 29 bp, synthetic construct
  source 1..29: mol_type = other DNA
aatgatacggcgaccaccgagatctacac

SEQ ID NO: 5 - DNA, 32 bp, synthetic construct
  source 1..32: mol_type = other DNA
gatcggaagagcgtcgtgtagggaaagagtgt

SEQ ID NO: 6 - DNA, 34 bp, synthetic construct
  source 1..34: mol_type = other DNA
gtgactggagttcagacgtgtgctcttccgatct

SEQ ID NO: 7 - DNA, 33 bp, synthetic construct
  source 1..33: mol_type = other DNA
gatcggaagagcacacgtctgaactccagtcac

SEQ ID NO: 8 - DNA, 33 bp, synthetic construct
  source 1..33: mol_type = other DNA
acactctttccctacacgacgctcttccgatct

SEQ ID NO: 9 - DNA, 23 bp, synthetic construct
  source 1..23: mol_type = other DNA
gggcctaddddddddtaggccct